(19) United States
(12) Patent Application Publication
(10) Pub. No.: US 2005/0271661 A1
(43) Pub. Date: Dec. 8, 2005

(54) Methods and reagents for the treatment of immunoinflammatory disorders

(75) Inventors: Palaniyandi Manivasakam, West Roxbury, MA (US); Edward Roydon Jost-Price, West Roxbury, MA (US); Jane Staunton, Roslindale, MA (US); Curtis Keith, Boston, MA (US)

Correspondence Address: CLARK & ELBING LLP, 101 FEDERAL STREET, BOSTON, MA, 02110, US

(73) Assignee: CombinatoRx, Incorporated, Boston, MA (US)

(21) Appl. No.: 11/130,426

(22) Filed: May 16, 2005

Related U.S. Application Data
(60) Provisional application No. 60/571,757, filed on May 17, 2004.

(51) Int. Cl. A61K 39/395; A61K 38/13; A61K 31/4745; A61K 31/192; A61K 31/522; A61K 31/59; A61K 31/415

(52) U.S. Cl. 424/144.1; 514/11; 514/291; 514/406; 514/569; 514/570; 514/167; 514/166; 514/454; 514/567; 514/649; 514/263.31

(57) ABSTRACT

The invention involves the treatment, prevention, and reduction of immunoinflammatory disorders involving the combination of an agent that increases the signal activity of a glucocorticoid receptor (e.g., glucocorticoid receptor agonist) and an agent that modulates the signaling activity of one or more signaling pathways selected from the NF-κB pathway, NFAT pathway, AP-1 pathway, and Elk-1 pathway such that proinflammatory cytokine secretion or production, or any other inflammatory response, is reduced. Further, screening methods are provided for identifying candidate compounds and strategies useful for treating, preventing, or reducing such conditions.

### CROSS-REFERENCE TO RELATED APPLICATIONS

This application claims benefit from U.S. Provisional Application No. 60/571,757, filed May 17, 2004, hereby incorporated by reference.

### FIELD OF THE INVENTION

In general, the present invention involves the treatment, prevention, and reduction of immunoinflammatory disorders. Further, screening methods are provided for identifying candidate compounds and strategies useful for treating, preventing, or reducing such conditions.

### BACKGROUND OF THE INVENTION

The invention relates to the treatment, prevention, or reduction of immunoinflammatory disorders.

Immunoinflammatory disorders are characterized by the inappropriate activation of the body's immune defenses. Rather than targeting infectious invaders, the immune response targets and damages the body's own tissues or transplanted tissues. The tissue targeted by the immune system varies with the disorder. For example, in multiple sclerosis, the immune response is directed against the neuronal tissue, while in Crohn's disease the digestive tract is targeted. Immunoinflammatory disorders affect millions of individuals and include conditions such as asthma, allergic intraocular inflammatory diseases, arthritis, atopic dermatitis, atopic eczema, diabetes, hemolytic anaemia, inflammatory dermatoses, inflammatory bowel or gastrointestinal disorders (e.g., Crohn's disease and ulcerative colitis), multiple sclerosis, myasthenia gravis, pruritis/inflammation, psoriasis, rheumatoid arthritis, cirrhosis, and systemic lupus erythematosus.

Current treatment regimens for immunoinflammatory disorders typically rely on immunosuppressive agents. The effectiveness of these agents can vary and their use is often accompanied by adverse side effects. Thus, improved therapeutic agents and methods for the treatment of immunoinflammatory disorders are needed.

### SUMMARY OF THE INVENTION

The invention features compositions, methods, and kits for treating, preventing, and reducing immunoinflammatory disorders.

In one aspect, the invention features a composition containing an agent that increases glucocorticoid receptor signaling activity (e.g., a glucocorticoid receptor agonist such as prednisolone and dexamethasone) and a non-steroidal agent that modulates the signaling activity of at least one (desirably two, three, or more) of the following signaling pathways: NF-κB pathway, NFAT pathway, AP-1 pathway, and Elk-1 pathway such that proinflammatory cytokine secretion or production or any other inflammatory response (e.g., chemokine production, expression of cell surface markers) is reduced. These agents are present in amounts that, when administered to a mammal, are sufficient to reduce proinflammatory cytokine secretion or production or any other inflammatory response. If desired, the agent that increases glucocorticoid receptor signaling activity is present in the composition in low dosage. The composition may be formulated for topical or systemic administration.

The invention also features a method for treating, preventing, or reducing an immunoinflammatory disorder by administering to a mammal a combination of an agent that increases the signaling activity of a glucocorticoid receptor and a non-steroidal agent that modulates the signaling activity of one or more of the following signaling pathways: NF-κB pathway, NFAT pathway, AP-1 pathway, and Elk-1 pathway such that proinflammatory cytokine secretion or production or any other inflammatory response is reduced. The first and second agents are administered simultaneously or within 28 days of each other, in amounts that together are sufficient to treat, prevent, or reduce the immunoinflammatory disorder. The two agents are desirably administered within 14 days of each other, more desirably within seven days of each other, and even more desirably within twenty-four hours of each other, or even simultaneously (i.e., concomitantly). If desired, the agent that increases glucocorticoid receptor signaling activity is administered in low dosage.

The invention further features a method of reducing the release from or production of inflammatory cytokines in inflammatory cells (e.g., T cells). This method involves contacting inflammatory cells with an agent that increases the signaling activity of the glucocorticoid receptor and a non-steroidal agent that modulates the signaling activity of one or more of the following signaling pathways: NF-κB pathway, NFAT pathway, AP-1 pathway, and Elk-1 pathway such that proinflammatory cytokine secretion or production or any other inflammatory response is reduced.

In all foregoing aspects of the invention, the non-steroidal agent may be an agent that increases or decreases the expression level or biological activity (e.g., enzymatic activity, phosphorylation state, or binding activity) of a signaling molecule such that the signaling activity of one or more of the one or more of the signaling pathways (e.g., NF-κB pathway, NFAT pathway, AP-1 pathway, and Elk-1 pathway) is modulated (e.g., increased or reduced). For example, the non-steroidal agent may be an NF-κB pathway modulator, NFAT pathway modulator, AP-1 pathway modulator, or Elk-1 pathway modulator. The non-steroidal agent may also be an antisense compound or RNAi compound that reduces the expression levels of a signaling molecule, such that the signaling activity of one or more of the signaling pathways (e.g., NF-κB pathway, NFAT pathway, AP-1 pathway, and Elk-1 pathway) is modulated. Alternatively, the non-steroidal agent may be a dominant negative form of a signaling molecule or an expression vector encoding a dominant negative such that the signaling activity of one or more of the NF-κB pathway, NFAT pathway, AP-1 pathway, or Elk-1 pathway is modulated. The non-steroidal agent may also be an antibody that binds a signaling molecule and reduces the biological activity of the signaling molecule such that the signaling activity of one or more of the NF-κB pathway, NFAT pathway, AP-1 pathway, and Elk-1 pathway is modulated. In addition, the non-steroidal agent may be an agent that affects chromatin conformation such as modulators of histone deacetylases (HDAC) or histone acetyl transferases. The non-steroidal agent may also be an inhibitor of pro-inflammatory cytokine mRNA stabilization complexes (e.g. TIA-1, TIAR, TTP) or pathways that lead to the activation of these complexes.

If desired, an additional therapeutic compound may be formulated or administered with the combination of the invention. This additional therapeutic compound may be, for example, an NSAID, small molecule immunomodulator, COX-2 inhibitor, DMARD, biologic, xanthine, anticholinergic compound, beta receptor agonist, bronchodilator, non-steroidal calcineurin inhibitor, vitamin D analog, psoralen, retinoid, or 5-amino salicylic acid.

The invention also features various screening methods to identify candidate compounds and strategies to treat, prevent, or reduce immunoinflammatory conditions. For example, one method for identifying a combination that may be useful for the treatment, prevention, or reduction of an immunoinflammatory disorder involves the steps of: (a) contacting inflammatory cells (e.g., T cells) in vitro with an agent that increases the signaling activity of the glucocorticoid receptor and a candidate compound; and (b) determining whether the combination of the agent that increases the signaling activity of the glucocorticoid receptor and the candidate compound reduces proinflammatory cytokine release from or production in these cells relative to proinflammatory cytokine release from or production in cells contacted with the agent that increases the signaling activity of the glucocorticoid receptor but not contacted with the candidate compound. A reduction in proinflammatory cytokine release or production identifies the combination as a combination useful for the treatment, prevention, or reduction of an immunoinflammatory disorder.

Another screening method for identifying a candidate compound useful for the treatment, prevention, or reduction of an immunoinflammatory disorder involves the steps of: (a) providing inflammatory cells having reduced glucocorticoid receptor signaling activity; (b) contacting these cells with a candidate compound; and (c) determining whether the candidate compound reduces cytokine release from or production in said cells relative to cells not contacted with the candidate compound. A reduction in cytokine release or production identifies the candidate compound as a compound useful for the treatment, prevention, or reduction of an immunoinflammatory disorder.

The invention also features a method for identifying a combination that may be useful for the treatment of an immunoinflammatory disorder, involving the steps of: (a) contacting inflammatory cells in vitro with an agent that increases the signaling activity of the glucocorticoid receptor and a candidate compound; and (b) determining whether the combination of the agent that increases the signaling activity of the glucocorticoid receptor and the candidate compound reduces cytokine release from or production in these inflammatory cells relative to cytokine release or production from cells contacted with the agent that increases the signaling activity of the glucocorticoid receptor but not contacted with the candidate compound. A reduction in cytokine release or production identifies the combination as a combination useful for the treatment, prevention, or reduction of an immunoinflammatory disorder.

The invention further features a method for identifying a compound useful for the treatment, prevention, or reduction of an immunomodulatory disorder, involving the steps of: (a) providing inflammatory cells engineered to have reduced signaling activity in one or more of the NF-κB pathway, NFAT pathway, AP-1 pathway, and Elk-1; (b) contacting these cells with a candidate compound; and (c) determining whether the candidate compound reduces proinflammatory cytokine release from or production in cells relative to cells not contacted with the candidate compound. A reduction in cytokine release or production identifies the candidate compound as a compound useful for the treatment, prevention, or reduction of an immunoinflammatory disorder.

The invention also features a method for identifying a combination useful for the treatment, prevention, or reduction of an immunoinflammatory disorder, involving the steps of: (a) identifying a compound that modulates signaling activity of one or more of the NF-κB pathway, NFAT pathway, AP-1 pathway, and Elk-1 pathway; (b) contacting inflammatory cells in vitro with an agent that increases the signaling activity of the glucocorticoid receptor and the compound identified in step (a); and (c) determining whether the combination of the agent that increases the signaling activity of the glucocorticoid receptor and the compound identified in step (a) reduces proinflammatory cytokine release from or production in said cells relative to cells contacted with said agent that increases the signaling activity of the glucocorticoid receptor but not contacted with the compound identified in step (a) or contacted with the compound identified in step (a) but not contacted with said agent that increases the signaling activity of the glucocorticoid receptor. A reduction in proinflammatory cytokine release or production identifies the combination as a combination useful for the treatment, prevention, or reduction of an immunoinflammatory disorder.

The invention also features a method for identifying a combination useful for the treatment, prevention, or reduction of an immunoinflammatory disorder, this method involving the steps of: (a) identifying a compound that modulates signaling activity of one or more of the NF-κB pathway, NFAT pathway, AP-1 pathway, and Elk-1 pathway such that proinflammatory cytokine secretion or production or any other inflammatory response is reduced; (b) contacting inflammatory cells in vitro with an agent that increases the signaling activity of a glucocorticoid receptor and the compound identified in step (a); and (c) determining whether the combination of these agents reduces proinflammatory cytokine release from or production in said cells relative to cytokine release from or production in cells contacted with the agent that increases the signaling activity of the glucocorticoid receptor but not contacted with the compound identified in step (a) or contacted with the compound identified in step (a) but not contacted with the agent that increases the signaling activity of the glucocorticoid receptor. A reduction in proinflammatory cytokine release identifies the combination as useful for the treatment, prevention, or reduction of an immunoinflammatory disorder.

The invention also features a kit containing: (i) a composition that contains an agent that increases the signaling activity of the glucocorticoid receptor and a non-steroidal agent that modulates the signaling activity of one or more of the NF-κB pathway, NFAT pathway, AP-1 pathway, and Elk-1 pathway such that proinflammatory cytokine secretion or production or any other inflammatory response is reduced; and (ii) instructions for administering this composition to a patient diagnosed with an immunoinflammatory disorder.

The invention also features a kit that contains (i) an agent that increases the signaling activity of the glucocorticoid receptor; (ii) a non-steroidal agent that modulates the signaling activity of one or more of the NF-κB pathway, NFAT pathway, AP-1 pathway, and Elk-1 pathway such that proinflammatory cytokine secretion or production or any other inflammatory response is reduced; and (iii) instructions for administering the agent that increases the signaling activity of the glucocorticoid receptor and the non-steroidal agent to a patient diagnosed with an immunoinflammatory disorder.

Another kit provided in the present invention contains (i) an agent that increases the signaling activity of the glucocorticoid receptor; and (ii) instructions for administering this agent and a non-steroidal agent that modulates the signaling activity of one or more of the NF-κB, NFAT, AP-1, and Elk-1 pathways such that proinflammatory cytokine secretion or production or any other inflammatory response is reduced to a patient diagnosed with an immunoinflammatory disorder.

Alternatively, the invention provides a kit containing (i) a non-steroidal agent that modulates the signaling activity of one or more of the NF-κB pathway, NFAT pathway, AP-1 pathway, and Elk-1 pathway such that proinflammatory cytokine secretion or production or any other inflammatory response is reduced; and (ii) instructions for administering this agent and an agent that increases the signaling activity of the glucocorticoid receptor to a patient diagnosed with an immunoinflammatory disorder.

By “treating, reducing, or preventing an immuinflammatory disorder” is meant ameliorating such condition before or after it has occurred. As compared with an equivalent untreated control, such reduction or degree of prevention is at least 5%, 10%, 20%, 40%, 50%, 60%, 80%, 90%, 95%, or 100% as measured by any standard technique. A patient who is being treated for an immunoinflammatory disorder is one who a medical practitioner has diagnosed as having such a condition. Diagnosis may be by any suitable means. One in the art will understand that these patients may have been subjected to the standard tests or may have been identified, without examination, as one at high risk due to the presence of one or more risk factors, such as family history.

By “patient” is meant any animal (e.g., a human). Other animals that can be treated using the methods, compositions, and kits of the invention include horses, dogs, cats, pigs, goats, rabbits, hamsters, monkeys, guinea pigs, rats, mice, lizards, snakes, sheep, cattle, fish, and birds.

By “a signaling pathway” is meant a series of intracellular molecular signals that are generated as a result of an external cellular stimulus, ultimately leading to the expression of specific effector proteins that elicit a cellular or biological effect (e.g., inflammation). For example, a ligand may bind a receptor at the cell surface, resulting in the recruitment and activation of various cellular proteins (e.g., protein kinases). Once these initial intracellular proteins are activated, the external signal is further propagated and amplified by the recruitment and activation of other intracellular proteins, leading to the transcription and expression of effector proteins (e.g., proinflammatory cytokines) that can elicit a biological or cellular phenotype (e.g., inflammation). The external stimuli may increase the expression of effector proteins in a cell by at least 10%, 20%, 30%, 40%, 50%, 60%, 70%, 80%, 90%, or 100% relative to a cell that has not been exposed to the external stimuli. Depending on the initiating stimuli, the biological activity or the expression level of intracellular signaling molecules within the signaling pathway may be increased or decreased by at least 10%, 20%, 30%, 40%, 50%, 60%, 70%, 80%, 90%, or 100% relative to such activity or expression in a control cell.

By “increasing the signaling activity of a glucocorticoid receptor” is meant to increase or decrease the expression level or biological activity of any of the signaling molecule involved in the signaling pathway of a glucocorticoid receptor. As a result, the signaling pathway downstream of this molecule is amplified and ultimately, the overall output of the glucocorticoid receptor signaling pathway is increased. Such increase in signaling activity may be the result of increasing or decreasing the expression level or biological activity of a signaling molecule in the signaling pathway by at least 10%, 20%, 30%, 0%, 50%, 60%, 70%, 80%, 90%, or 100% relative to an untreated control, as measured by any standard technique known in the art or described herein.

By “reducing the signaling activity of a signaling pathway” is meant to reduce the expression level or biological activity of any of the signaling molecule in the signaling pathway, thereby interfering with the propagation of the signaling pathway downstream of such molecule and ultimately, the overall output of the signaling pathway. Such reduction may be the result of increasing or decreasing the expression level or biological activity of a signaling molecule in the signaling pathway by at least 10%, 20%, 30%, 0%, 50%, 60%, 70%, 80%, 90%, or 100% relative to an untreated control, as measured by any standard technique known in the art or described herein. Ultimately, by reducing the signaling activity of a signaling pathway (e.g., one or more of the NFκB, NFAT, AP-1, or Elk-1 pathways), the expression of effector proteins (e.g., proinflammatory cytokines) is reduced by at least 10%, 20%, 30%, 40%, 50%, 60%, 70%, 80%, 90%, or 100% relative to a control cell. Alternatively, the biological output of the signaling pathway, such as the release or production of proinflammatory cytokines, is reduced by at least 10%, 20%, 30%, 40%, 50%, 60%, 70%, 80%, 90%, or 100% relative to a control.

In addition to increasing the signaling activity of the glucocorticoid receptor pathway, the treatment, prevention, or reduction of immunoinflammatory disorders according to this invention is achieved by modulating the signaling activity of one or more the signaling pathways involved in the production of the following effector proteins or transcription factors: NFκB, NFAT, AP-1, and Elk-1 such that proinflammatory cytokine secretion or production or any other inflammatory response is reduced. Such modulation may result from the increase or reduction of the expression level or biological activity of any of the signaling molecules involved in such pathways (as shown in FIG. 1) or by the modulation of any of the signaling activities depicted in FIG. 1. For example, the signaling activity of the NFAT signaling pathway may be reduced by interfering or reducing one or more of the following activities: calcium flux, calmodulin activation, calcineurin activation, NFAT dephosphorylation, NFAT translocation, or NFAT transcriptional activation. The signaling activity of the NFκB pathway may be reduced by inhibiting or reducing PKC activation, NIK activation, IKK activation, IκB phosphorylation and destruction, NFκB translocation, NFκB DNA binding, NFκB phosphorylation (on p65), and NFκB transcriptional activation. The signaling activity of AP-1 may be reduced by reducing one or more of the following: PKC activation, MLK phosphorylation, MAP kinase phosphorylation and activation (e.g., MMKK3/6 phosphorylation, JNK1/2 phosphorylation, MEKK4 phosphorylation, MKK4/7 phosphorylation, p38 phosphorylation, Raf phosphorylation, MEK1/2 phosphorylation, ERK1/2 phosphorylation, and cJun phosphorylation), AP-1 DNA binding, and AP-1 transcriptional activation. The signaling events and signaling molecules that may be modulated such that at least one of the NFAT, NFκB, AP-1, and Elk-1 pathways are reduced are shown, for example, in FIG. 1. Because the NFκB pathway, the NFAT pathway, the AP-1 pathway, and the Elk-1 pathway can increase proinflammatory cytokine release or production, the modulation of one or more these pathways results in the treatment, prevention, or reduction of immunoinflammatory disorders.

By “an amount sufficient” is meant the amount of a compound, in a combination of the invention, required to treat or prevent an immunoinflammatory disease in a clinically relevant manner. A sufficient amount of an active compound used to practice the present invention for therapeutic treatment of conditions caused by or contributing to an immunoinflammatory disease varies depending upon the manner of administration, the age, body weight, and general health of the mammal or patient. Ultimately, the prescribers will decide the appropriate amount and dosage regimen. Additionally, an effective amount may can be that amount of compound in the combination of the invention that is safe and efficacious in the treatment of a patient having the immunoinflammatory disease over each agent alone as determined and approved by a regulatory authority (such as the U.S. Food and Drug Administration).

By “more effective” is meant that a treatment exhibits greater efficacy, or is less toxic, safer, more convenient, or less expensive than another treatment with which it is being compared. Efficacy may be measured by a skilled practitioner using any standard method that is appropriate for a given indication.

The term “immunoinflammatory disorder” encompasses a variety of conditions, including autoimmune diseases, proliferative skin diseases, and inflammatory dermatoses. Immunoinflammatory disorders result in the destruction of healthy tissue by an inflammatory process, dysregulation of the immune system, and unwanted proliferation of cells. Examples of immunoinflammatory disorders are acne vulgaris; acute respiratory distress syndrome; Addison's disease; allergic rhinitis; allergic intraocular inflammatory diseases, ANCA-associated small-vessel vasculitis; ankylosing spondylitis; arthritis, asthma; atherosclerosis; atopic dermatitis; autoimmune hepatitis; autoimmune hemolytic anemia; autoimmune hepatitis; Behcet's disease; Bell's palsy; bullous pemphigoid; cerebral ischaemia; chronic obstructive pulmonary disease; cirrhosis; Cogan's syndrome; contact dermatitis; COPD; Crohn's disease; Cushing's syndrome; dermatomyositis; diabetes mellitus; discoid lupus erythematosus; eosinophilic fasciitis; erythema nodosum; exfoliative dermatitis; fibromyalgia; focal glomerulosclerosis; focal segmental glomerulosclerosis; giant cell arteritis; gout; gouty arthritis; graft-versus-host disease; hand eczema; Henoch-Schonlein purpura; herpes gestationis; hirsutism; idiopathic cerato-scleritis; idiopathic pulmonary fibrosis; idiopathic thrombocytopenic purpura; immune thrombocytopenic purpura inflammatory bowel or gastrointestinal disorders, inflammatory dermatoses; lichen planus; lupus nephritis; lymphomatous tracheobronchitis; macular edema; multiple sclerosis; myasthenia gravis; myositis; nonspecific fibrosing lung disease; osteoarthritis; pancreatitis; pemphigoid gestationis; pemphigus vulgaris; periodontitis; polyarteritis nodosa; polymyalgia rheumatica; pruritus scroti; pruritis/inflammation, psoriasis; psoriatic arthritis; pulmonary histoplasmosis; rheumatoid arthritis; relapsing polychondritis; rosacea caused by sarcoidosis; rosacea caused by scleroderma; rosacea caused by Sweet's syndrome; rosacea caused by systemic lupus erythematosus; rosacea caused by urticaria; rosacea caused by zoster-associated pain; sarcoidosis; scleroderma; segmental glomerulosclerosis; septic shock syndrome; shoulder tendinitis or bursitis; Sjogren's syndrome; Still's disease; stroke-induced brain cell death; Sweet's disease; systemic lupus erythematosus; systemic sclerosis; Takayasu's arteritis; temporal arteritis; toxic epidermal necrolysis; transplant-rejection and transplant-rejection-related syndromes; tuberculosis; type-1 diabetes; ulcerative colitis; uveitis; vasculitis; and Wegener's granulomatosis.

“Non-dermal inflammatory disorders” include, for example, rheumatoid arthritis, inflammatory bowel disease, asthma, and chronic obstructive pulmonary disease.

“Dermal inflammatory disorders” or “inflammatory dermatoses” include, for example, psoriasis, acute febrile neutrophilic dermatosis, eczema (e.g., asteatotic eczema, dyshidrotic eczema, vesicular palmoplantar eczema), balanitis circumscripta plasmacellularis, balanoposthitis, Behcet's disease, erythema annulare centrifugum, erythema dyschromicum perstans, erythema multiforme, granuloma annulare, lichen nitidus, lichen planus, lichen sclerosus et atrophicus, lichen simplex chronicus, lichen spinulosus, nummular dermatitis, pyoderma gangrenosum, sarcoidosis, subcorneal pustular dermatosis, urticaria, and transient acantholytic dermatosis.

By “proliferative skin disease” is meant a benign or malignant disease that is characterized by accelerated cell division in the epidermis or dermis. Examples of proliferative skin diseases are psoriasis, atopic dermatitis, non-specific dermatitis, primary irritant contact dermatitis, allergic contact dermatitis, basal and squamous cell carcinomas of the skin, lamellar ichthyosis, epidermolytic hyperkeratosis, premalignant keratosis, acne, and seborrheic dermatitis.

As will be appreciated by one skilled in the art, a particular disease, disorder, or condition may be characterized as being both a proliferative skin disease and an inflammatory dermatosis. An example of such a disease is psoriasis.

By a “low dosage” is meant at least 5% less (e.g., at least 10%, 20%, 50%, 80%, 90%, or even 95%) than the lowest standard recommended dosage of a particular compound formulated for a given route of administration for treatment of any human disease or condition. For example, a low dosage of an agent that increases the signaling activity of a glucocorticoid receptor formulated for administration by inhalation will differ from a low dosage of the same agent formulated for oral administration.

By a “high dosage” is meant at least 5% (e.g., at least 10%, 20%, 50%, 100%, 200%, or even 300%) more than the highest standard recommended dosage of a particular compound for treatment of any human disease or condition.

By a “candidate compound” is meant a chemical, be it naturally-occurring or artificially-derived. Candidate compounds may include, for example, peptides, polypeptides, synthetic organic molecules, naturally occurring organic molecules, nucleic acid molecules, peptide nucleic acid molecules, and components and derivatives thereof.

Compounds useful in the invention include those described herein in any of their pharmaceutically acceptable forms, including isomers such as diastereomers and enantiomers, salts, esters, solvates, and polymorphs thereof, as well as racemic mixtures and pure isomers of the compounds described herein.

By “corticosteroid” is meant any naturally occurring or synthetic compound characterized by a hydrogenated cyclopentanoperhydrophenanthrene ring system and having immunosuppressive and/or antinflammatory activity. Naturally occurring corticosteriods are generally produced by the adrenal cortex. Synthetic corticosteriods may be halogenated. Examples corticosteroids are provided herein.

By “non-steroidal immunophilin-dependent immunosuppressant” or “NsIDI” is meant any non-steroidal agent that decreases proinflammatory cytokine production or secretion, binds an immunophilin, or causes a down regulation of the proinflammatory reaction. NsIDIs include calcineurin inhibitors, such as cyclosporine, tacrolimus, ascomycin, pimecrolimus, as well as other agents (peptides, peptide fragments, chemically modified peptides, or peptide mimetics) that inhibit the phosphatase activity of calcineurin. NsIDIs also include rapamycin (sirolimus) and everolimus, which bind to an FK506-binding protein, FKBP-12, and block antigen-induced proliferation of white blood cells and cytokine secretion.

By “small molecule immunomodulator” is meant a non-steroidal, non-NsIDI compound that decreases proinflammatory cytokine production or secretion, causes a down regulation of the proinflammatory reaction, or otherwise modulates the immune system in an immunophilin-independent manner. Examplary small molecule immunomodulators are p38 MAP kinase inhibitors such as VX 702 (Vertex Pharmaceuticals), SCIO 469 (Scios), doramapimod (Boehringer Ingelheim), RO 30201195 (Roche), and SCIO 323 (Scios), TACE inhibitors such as DPC 333 (Bristol Myers Squibb), ICE inhibitors such as pranalcasan (Vertex Pharmaceuticals), and IMPDH inhibitors such as mycophenolate (Roche) and merimepodib (Vertex Pharamceuticals).

Other features and advantages of the invention will be apparent from the detailed description and from the claims.

### BRIEF DESCRIPTION OF THE DRAWINGS

FIG. 1 is a schematic diagram depicting the NFκB, NFAT, Elk-1, and AP-1 signaling pathway.

FIGS. 2A-2C are a series of illustrations showing amoxapine and paroxetine repress the NFAT pathway. T cells were activated with PMA (90 ng/ml)/ionomycin (5 μg/ml) with or without increasing amount of the test drugs amoxapine, paroxetine, prednisolone and cyclosporine. FIG. 2A shows T cell line CCRF-CEM transfected with a NFAT luciferase reporter four hours before preincubation with vehicle or drug treatment (n=4 experiment). FIG. 2B shows western blots of primary T cells purified and processed with NFAT1-specific antibodies. FIG. 2C shows nuclear translocation analysis of T cell line CCRF-CEM drug-treated for 20 minutes and stimulated thereafter for one hour and processed for immunofluorescence.

FIGS. 3A-3C are a series of illustrations showing amoxapine and paroxetine repress the NF-κB pathway. T cells were activated with PMA/ionomycin with or without increasing amount of the test drugs amoxapine, paroxetine, prednisolone, cyclosporine or CAPE. FIG. 3A shows the results of T cell line CCRF-CEM that were transfected with a NF-κB luciferase reporter 4 hours before preincubation with vehicle or drug treatment. (n=4 experiment) FIG. 3B shows western blot results from primary T cells purified and processed with NFAT1-specific antibodies. FIG. 3C shows nuclear translocation analysis of T cell line CCRF-CEM processed for immunofluorescence.

FIGS. 4A-4D are illustrations showing amoxapine and paroxetine repress the AP1 pathway. Primary T cells were preincubated with vehicle or drug 30 minutes before activation with PMA and ionomycin. Cells were extracted for 30 minutes later and processed for western blot analysis with phospospecific and antibodies that recognize total protein. FIG. 4A: ERK; FIG. 4B: p38; FIG. 4C: JNK). The blots were probed with alpha tubulin as a loading control. FIG. 4D is an illustration depicting AP1-dependent transcription measured by transient transfection of an AP1 reporter plasmid into CCRF-CEM cells and subsequent activation with PI.

### DETAILED DESCRIPTION

Despite their efficacy, the chronic use of glucocorticoids for treating immunoinflammatory disorders is often associated with serious systemic side effects. Although extensive efforts have been made to widen the steroid therapeutic window through structural modification of the steroid molecule, this approach has met with mixed success. Here, we have developed the first high-throughput platform for the discovery of ‘syncretic’ therapeutics involving combinations of compounds that interact synergistically to enhance therapeutic effects while minimizing debilitating side effects.

The invention features methods, compositions, and kits for the administration of an effective amount of an agent that increases the signaling activity of a glucocorticoid receptor (e.g., a glucocorticoid receptor agonist) in combination with an agent that modulates the signaling activity of one or more of the NF-κB pathway, NFAT pathway, AP-1 pathway, and Elk-1 pathway such that proinflammatory cytokine secretion or production or any other inflammatory response is reduced. Based on this invention, the administration of this combination causes a reduction in inflammation by reducing the production or release of pro-inflammatory chemokines or cytokines, such as TNF-α, thereby resulting in the treatment, prevention, and reduction of immunoinflammatory disorders. Desirably, the agent that increases the signaling activity of a glucocorticoid receptor is formulated or administered with an agent that modulates the signaling activity of more than one of the NFκB, NFAT, AP-1, and Elk-1 pathways such that proinflammatory cytokine secretion or production or any other inflammatory response is reduced (e.g., an agent that modulates the signaling activity of the NFκB and NFAT signaling pathways).

The compositions, methods, and kits of the invention are useful for treating, preventing, or reducing an immunoinflammatory disorder, proliferative skin disease, organ transplant rejection, or graft versus host disease. The combination of multiple agents may also be desirable. For example, methotrexate, hydroxychloroquine, and sulfasalazine are commonly administered for the treatment of rheumatoid arthritis and may therefore be administered with the combinations described herein.

The invention is described in greater detail below.

Agents Increasing Glucocorticoid Receptor Signaling Activity

Agents that increase the signaling activity of a glucocorticoid receptor are used in combination with an agent that reduces the signaling activity of one or more of the following pathways: NF-κB pathway, NFAT pathway, AP-1 pathway, and Elk-1 pathway in the methods, compositions, and kits of the invention. Agents that increase the signaling activity of a glucocorticoid receptor ultimately increase glucocorticoid receptor-driven transcription. Such an increase in activity may result, for example, by increasing one or more of the following activities: receptor binding, receptor/GC translocation, receptor/GC DNA binding, receptor/GC transcriptional activation, or receptor/GC transrepression. Exemplary agents that may used in the methods, compositions, and kits of the invention include compounds described in U.S. Pat. Nos. 6,380,207, 6,380,223, 6,448,405, 6,506,766, and 6,570,020, U.S. Patent Application Publication Nos. 20030176478, 20030171585, 20030120081, 20030073703, 2002015631, 20020147336, 20020107235, 20020103217, and 20010041802, and PCT Publication No. WO00/66522, each of which is hereby incorporated by reference. Other agents that may also be used in the methods, compositions, and kits of the invention are described in U.S. Pat. Nos. 6,093,821, 6,121,450, 5,994,544, 5,696,133, 5,696,127, 5,693,647, 5,693,646, 5,688,810, 5,688,808, and 5,696,130, each of which is hereby incorporated by reference.

Agents that Modulate the Signaling Activity of NF-κB Pathway, NFAT Pathway, AP-1 Pathway, and Elk-1 Pathway

The agent that increases the signaling activity of a glucocorticoid receptor is formulated or administered with a non-steroidal agent that modulates the signaling activity of one or more of the NFκB, NFAT, Elk-1, and AP-1 pathways such that proinflammatory cytokine secretion or production or any other inflammatory response is reduced. This non-steroidal agent may increase or reduce the expression level or biological activity of any one of the signaling molecules in these pathways, such that the end-result is a modulation in the signaling activity of one or more of NFκB, NFAT, Elk-1, and AP-1 signaling pathways. Useful agents are described, for example, in Palanki, Curr. Med. Chem. 9:219-27 (2002).

Agents that Modulate the Signaling Activity of NFκB Pathway

Agents that modulate the signaling activity of the NFκB signaling pathway may modulate, for example, one or more of the following activities: PKC activation, NIK activation, IKK activation, IκB phosphorylation and destruction, NFκB translocation, NFκB DNA binding, NFκB phosphorylation (p65) or NFκB transcriptional activation. These compounds are described, for example, in U.S. Patent Application Publication Nos. 20040092430, 20040058930, and 20030013170, 20030078246 and 20030078246, and U.S. Ser. No. 10/670,488, filed Sep. 24, 2003, all of which are hereby incorporated by reference. Such agents include α-lipoic acid, α-tocopherol, anetholdithiolthione (ADT), astaxanthin, bis-eugenol, butylated hydroxyanisole (BHA), cepharanthine caffeic acid phenethyl ester (3,4-dihydroxycinnamic acid, CAPE), carnosol, carvedilol, catechol derivatives, durcumin (diferulolylmethane), dibenzylbutyrolactone lignans, diethyldithiocarbamate (DDC), iferoxamine, dihydrolipoic Acid, dilazep with fenofibric acid, dimethyldithiocarbamates (DMDTC), curcumin (diferulolylmethane), disulfiram, ebselen, EPC-K1 (phosphodiester compound of vitamin E and vitamin C), epigallocatechin-3-gallate (EGCG; green tea polyphenols), ergothioneine, ethyl pyruvate, ethylene glycol tetraacetic acid (EGTA), gamma-glutamylcysteine synthetase (gamma-GCS), ganoderma lucidum polysaccharides, ginkgo biloba extract, glutathione, hematein, IRFI 042 (vitamin E-like compound), ron tetrakis, lacidipine, lazaroids, lupeol, magnolol, manganese superoxide dismutase (Mn-SOD), N-acetyl-L-cysteine (NAC), nacyselyn (NAL), nordihydroguaiaritic acid (NDGA), orthophenanthroline, phenolic antioxidants (e.g., hydroquinone and tert-butyl hydroquinone), phenylarsine oxide (PAO, tyrosine phosphatase inhibitor), pyrrolinedithiocarbamate (PDTC), quercetin, Rg(3) (a ginseng derivative), rotenone, S-allyl-cysteine (SAC), sauchinone, tepoxaline (5-(4-chlorophenyl)-N-hydroxy-(4-methoxyphenyl)-N-methyl-1H-pyrazole-3-propanamide), α-torphryl succinate, α-torphryl acetate, PMC (2,2,5,7,8-pentamethyl-6-hydroxychromane), and yakuchinone A and B. NFκB inhibitors also include proteosome inhibitors, such as peptide aldehydes (ALLnL(N-acetyl-leucinyl-leucynil-norleucynal, MG101), LLM (N-acetyl-leucinyl-leucynil-methional), Z-LLnV, (carbobenzoxyl-leucinyl-leucynil-norvalinal, MG115), Z-LLL (carbobenzoxyl-leucinyl-leucynil-leucynal, MG132), lactacystine, b-lactone, boronic acid peptide, ubiquitin ligase inhibitors, PS-341, cyclosporin A, FK506 (tacrolimus), deoxyspergualin, APNE (N-acetyl-DL-phenylalanine-b-naphthylester), BTEE (N-benzoyl L-tyrosine-ethylester), DCIC (3,4-dichloroisocoumarin), DFP (diisopropyl fluorophosphate), TPCK (N-a-tosyl-L-phenylalanine chloromethyl ketone), calagualine (fern derivative), LY29 and LY30, pefabloc (serine protease inhibitor), rocaglamides (aglaia derivatives), geldanamycin, BMS-345541 (4(2′-Aminoethyl)amino-1,8-dimethylimidazo[1,2-a) quinoxaline), 2-amino-3-cyano-4-aryl-6-(2-hydroxy-phenyl)pryridine analog (compoud 26), anandamide, AS602868, BMS-345541, flavopiridol, jesterone dimer, apigenin, HB-EGF (Heparin-binding epidermal growth factor-like growth factor, LF15-0195 (analog of 15-deoxyspergualine), MX781 (retinoid antagonist), itrosylcobalamin (vitamin B12 analog), survanta, PTEN (tumor suppressor), silibinin, sulfasalazine, piceatannol, quercetin, staurosporine, wedelolactone, betulinic acid, ursolic acid, anethole, aspirin, sodium salicylate, azidothymidine (AZT), BAY-117082, (E3((4-methylphenyl)-sulfonyl)-2-propenenitrile), BAY-117083, (E3((4-t-butylphenyl)-sulfonyl)-2-propenenitrile), benzyl isothiocyanate, cacospongionolide B, calagualine, carboplatin, chorionic gonadotropin, cycloepoxydon; 1-hydroxy-2-hydroxymethyl-3-pent-1-enylbenzene, digitoxin, 4-Hydroxynonenal (HNE), gabexate mesilate, glossogyne tenuifolia, hydroquinone, ibuprofen, indirubin-3′-oxime, nterferon-alpha, methotrexate, monochloramine, nafamostat mesilate, oleandrin, panduratin A, petrosaspongiolide M, phytic acid (inositol hexakisphosphate), prostaglandin A1, 20(S)-protopanaxatriol (ginsenoside metabolite), sanguinarine (pseudochelerythrine, 13-methyl-[1,3]-benzodioxolo-[5,6-c]-1,3-dioxolo-4,5 phenanthridinium), silymarin, SOCS1, sulindac, THI 52 (1-naphthylethyl-6,7-dihydroxy-1,2,3,4-tetrahydroisoquinoline), vesnarinone, YopJ (encoded by Yersinia pseudotuberculosis) acetaminophen, α-melanocyte-stimulating hormone (α-MSH), amentoflavone, artemisia capillaris thunb extract, aucubin, beta-lapachone, capsaicin (8-methyl-N-vanillyl-6-nonenamide), core protein of Hepatitis C virus (HCV), cyclolinteinone (sponge sesterterpene), diamide (tyrosine phosphatase inhibitor), E-73 (cycloheximide analog), ecabet sodium, emodin (3-methyl-1,6,8-trihydroxyanthraquinone), erbstatin (tyrosine kinase inhibitor), fosfomycin, fungal gliotoxin, gabexate mesilate, genistein (tyrosine kinase inhibitor), glimepiride, glucosamine sulfate, gamma-glutamycysteine synthetase, hypochlorite, isomallotochromanol, isomallotochromene, K1L (Vaccinia virus protein), Kochia scoparia fruit (methanol extract), leflunomide metabolite (A77 1726), losartin, LY294002 [2-(4-morpholinyl)-8-phenylchromone], 5′-methylthioadenosine, U0126, pervanadate, phenylarsine oxide (PAO, tyrosine phosphatase inhibitor), prostaglandin 15-deoxy-Delta(12,14)-PGJ(2), resiniferatoxin, sesquiterpene lactones (parthenolide; ergolide; guaianolides), thiopental, TNP-470, triglyceride-rich lipoproteins, epoxyquinone A monomer, Ro106-9920, conophylline MOL 294 (small molecule), rhein, apigenin (4′,5,7-trihydroxyflavone), dioxin, astragaloside IV, atorvastatin, dehydroxymethylepoxyquinomicin (DHMEQ), 15-deoxyspergualin, nucling o,o′-bismyristoyl thiamine disulfide (BMT), nicotinamide, 3-aminobenzamide, 7-amino-4-methylcoumarin, amrinone, angiopoietin-1, artemisinin, atrovastat, baicalein (5,6,7-trihydroxyflavone), benfotiamine biliverdin, bisphenol A, campthothecin, caprofin, capsiate, catalposide, diarylheptanoid 7-(4′-hydroxy-3′-methoxyphenyl)-1-phenylhept-4-en-3-one, DTD (4,10-dichloropyrido[5,6:4,5]thieno[3,2-d′: 3,2-d]-1,2,3-ditriazine), E3330 (quinone derivative), epoxyquinol A, flunixin meglumine, flurbiprofen, pentoxifylline (1-(5′-oxohexyl) 3,7-dimetylxanthine, PTX), 6(5H)-phenanthridinone and benzamide, phenyl-N-tert-butylnitrone (PBN), pirfenidone, pyrithione, quinadril raxofelast, rebamipide, ribavirin, rifamides, eolipram, sanggenon C, SUN C8079, T-614, tyrphostin AG-126, APC0576, D609, cycloprodigiosin hycrochloride, pranlukast, psychosine, quinazolines, resveratrol, RO31-8220, saucemeol D and saucemeol E, tranilast [N-(3,4-dimethoxycinnamoyl)anthranilic acid], 3,4,5-trimethoxy-4′-fluorochalcone, triptolide, mesalamine, 17-allylamino-17-demethoxygeldanamycin, 6-aminoquinazoline derivatives, luteolin, tetrathiomolybdate, trilinolein, troglitazone, wortmannin, and rifampicin. Agents that reduce any of the MAP kinases may also reduce NFκB signaling activity and are further described below.

Agents that Modulate the Signaling Activity of NFAT Pathway

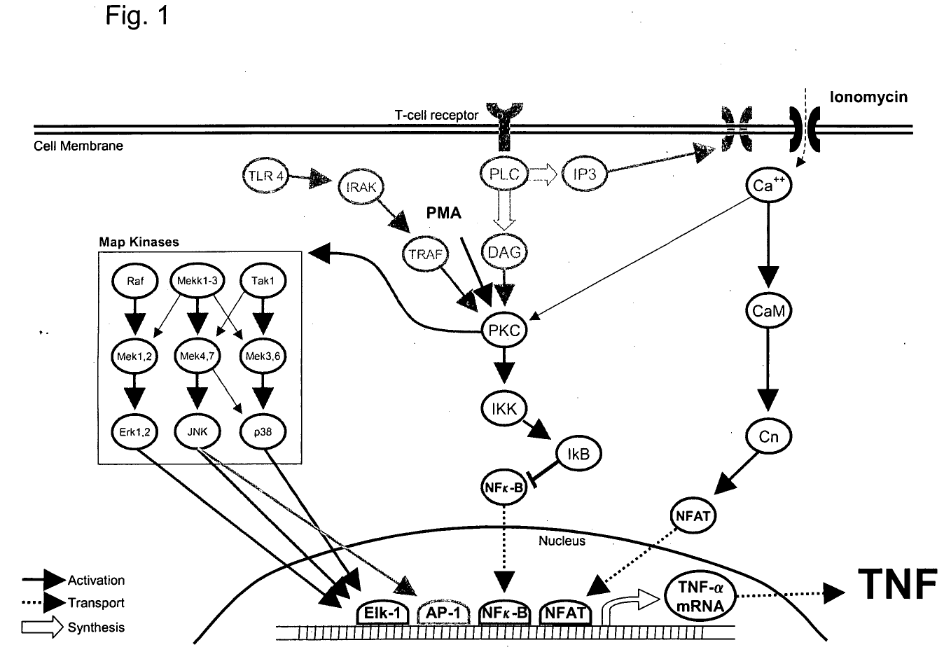

The calcium-sensitive phosphatase calcineurin is implicated in various biological systems including lymphocyte activation. As substrates of calcineurin, transcription factors of the NFAT family play an essential role in lymphocyte activation. Agents that modulate signaling activity of the NFAT signaling pathway can be divided into two class, protein inhibitors and small molecule inhibitors. Some of these inhibitors bind calcineurin and suppress dephosphorylating activity. For example, agents that modulate NFAT-driven transcription include agents that modulate any one of the following activities: calcium flux, calmodulin activation, calcineurin activation, NFAT dephosphorylation, NFAT translocation, or NFAT transcriptional activation (see FIG. 1). Protein inhibitors that prevent NFAT nuclear translocation include AKAP79, a scaffold protein that prevents calcineurin substrate interactions; CABIN protein, which blocks calcineurin activity; a calcineurin B homolog, CHP; and MCIP1, 2, 3 proteins which have the ability to prevent NFAT2 phosphorylation and nuclear import. NFAT small molecule inhibitors include cyclosporin A and FK506. Mechanistically, cyclosporin A and FK506 indirectly repress NFAT by inhibiting calcineurin activity. These agents target NFAT specific pathways and act as immunosuppressants by inhibiting alloreactive T-cells. Other agents include A-285222, D-43787, and 3,5-bistriflouromethylpyrazole (BTP) derivatives that inhibit Th1 and Th2 cytokine gene expression, thereby indirectly inhibiting the nuclear localization of NFAT. Other exemplary agents that reduce NFAT signaling are described by Martinez-Martinez et al., Current Medicinal Chemistry 11: 997-1007, in U.S. Patent Application Publication Nos. 20040002117 and 2002013230 as well as PCT WO03/0103647. Agents that modulate any of the MAP kinases may also modulate NFAT signaling activity and are further described below.

Agents that Modulate the Signaling Activity of AP-1 and Elk-1 Pathways

The glucocorticoid receptor agonist of the invention may be administered with an agent that modulates the signaling activity of the AP-1 signaling pathway, the Elk-1 signaling pathway, or both. Such an agent may modulates one or more of the following activities: PKC activation, MLK phosphorylation, activation and/or phosphorylation of a MAP kinase (e.g., Raf, MEK1/2, Erk1/2, MEKK1-3, MEK4/7, JNK1/2, Tak1, MEK3/6, or p38), DNA binding activity, or AP-1 transcriptional activation. Agents that modulates any of the MAP kinases may also modulates AP-1 and Elk-1 signaling activity and are further described below.

MAP Kinase Inhibitors

Because the family of MAP kinase proteins are central to the NFκB, NFAT, AP-1, and Elk-1 signaling pathways, any agent that modulates the phosphorylation state, activation, or both of a MAP kinase protein is useful in any of the combinations described herein. Thus, any inhibitor of the Raf, Mek1/2, ERK1/2, MEKK1/3, MEK4/7, JNK, p38, MEK3/6, Tak1 proteins may be used, for example, with the agent that increases the signaling activity of the glucocorticoid receptor. Agents that modulates signaling activity of MAP kinase proteins are described, for example, by Ravingerova et al., Mol. Cell Biochem. 247:127-38 (2003) and Chang et al., Leukemia. 17:1263-93 (2003). MEK inhibitors are described, for example, in U.S. Patent Application Publication No. 20040087583. Erk Kinase Inhibitors are described, for example, in U.S. Patent Application Publication Nos. 20040082631, 20040048861, 20040029857, 20030225151, 20030195241, 20030049820, 20020151574, 20030158238, 20030092714, 20030040536, and 20020177618. Erk Kinase inhibitors are further described by Rubinfeld et al., Methods Mol. Biol. 250:1-28 (2004) and Kohno et al., Prog. Cell Cycle Res. 5:219-24 (2003). Agents that modulate signaling activity of the Raf signaling pathway are described, for example, by Bollag et al., Curr. Opin. Invest. Drugs. 4:1436-41 (2003).

P38 Inhibitors

N-(3-tert-butyl-1-methyl-5-pyrazolyl)-N′-(4-(4-pyridinylmethyl)phenyl)urea, RPR 200765A, SB203580, SB202190, UX-745, UX-702, UX-850, and SC10-469 are exemplary p38 inhibitors. Other p38 inhibitors are described in U.S. Pat. Nos. 5,716,972, 5,686,455, 5,656,644, 5,593,992, 5,593,991, 5,663,334, 5,670,527, 5,559,137, 5,658,903, 5,739,143, 5,756,499, 5,716,955, WO 98/25619, WO 97/25048, WO 99/01452, WO 97/25047, WO 99/01131, WO 99/01130, WO 97/33883, WO 97/35856, WO 97/35855, WO 98/06715, WO 98/07425, WO 98/28292, WO 98/56377, WO 98/07966, WO 99/01136, WO 99/17776, WO 99/01131, WO 99/01130, WO 99/32121, WO 00/26209, WO 99/58502, WO 99/58523, WO 99/57101, WO 99/61426, WO 99/59960, WO 99/59959, WO 00/18738, WO 00/17175, WO 99/17204, WO 00/20402, WO 99/64400, WO 00/01688, WO 00/07980, WO 00/07991, WO 00/06563, WO 00/12074, WO 00/12497, WO 00/31072, WO 00/31063, WO 00/23072, WO 00/31065, WO 00/35911, WO 00/39116, WO 00/43384, WO 00/41698, WO 97/36587, WO 97/47618, WO 97/16442, WO 97/16441, WO 97/12876, WO 98/7966, WO 98/56377, WO 98/22109, WO 98/24782, WO 98/24780, WO 98/22457, WO 98/52558, WO 98/52941, WO 98/52937, WO 98/52940, WO 98/56788, WO 98/27098, WO 99/00357, WO 98/47892, WO 98/47899, WO 99/03837, WO 99/01441, WO 99/01449, WO 99/03484, WO 95/09853, WO 95/09851, WO 95/09847, WO 95/09852, WO 92/12154, WO 94/19350, WO 99/15164, WO 98/50356, DE 19842833, JP 2000 86657, and U.S. Patent Application Publication Nos. 20040092547, 20040082551, 20040077682, 20040077647, 20040053923, 20040053958, 20040053942, 20040044044, 20040023992, 20030216446, 20030203905, 20030195355, 20030149041, 20030149037, 20030144529, 20030144520, 20030139462, 20030134888, 20030130319, 20030100756, 20030100588, 20030096817, 20030092717, 20030083327, 20030078432, 20030078275, 20030078166, 20030073687, 20030064982, 20030064981, 20030055068, 20030055044, 20030036543, 20030004164, 20030004161, 20020156114, 20020156081, 20020115671, 20020103245, 20020086869, 20020019393, 20020016477, 20020013354, 20020010170, 20010025044, and 20010044538. p38 inhibitors are also described in Rupert et al., Bioorg Med Chem Lett. 13:347-50 (2003); Dumas et al., Bioorg Med Chem Lett. 12:1559-1562 (2002); Dumas et al., Bioorg Med Chem Lett. 10:2051-2054 (2000); Redman et al., Bioorg Med Chem Lett. 11:9-12 (2001); Wan et al., Bioorg Med Chem Lett. 13:1191-4 (2003); Regan et al., J Med. Chem. 45:2994-3008 (2002); Liverton et al., J Med. Chem. 42:2180-90 (1999); Dumas, Curr. Opin. Drug Discov. Devel. 5:718-27 (2002); Stelmach et al., Bioorg. Med. Chem. Lett. 13:277-80 (2003); Cirillo et al., Curr. Top. Med. Chem. 2:1021-35 (2002); Pargellis et al., Curr. Opin. Investig. Drugs. 4:566-71; Dumas et al., Bioorg. Med. Chem. Lett. 10:2047-50 (2000); Trejo et al., J. Med. Chem. 46:4702-13 (2003); Mclay et al. Bioorg. Med. Chem. 9:537-54 (2001); Lee et al., Immunopharmacology 47:185-201 (2000); Adams et al., Bioorg. Med. Chem. Lett. 11: 2867-70 (2001); Regan et al., J. Med. Chem. 46:4676-4686 (2003); Laufer et al., J. Med. Chem. 45:2733-40 (2002); Colletti et al., J. Med. Chem. 46:349-52 (2003), Branger et al., J. Immunol. 168:4070-7 (2002), Henry et al., Bioorg. Med. Chem. Lett. 8:3335-40 (1998); Adams et al., Prog. Med. Chem. 38:1-60 (2001), Revesz et al., Bioorg. Med. Chem. Lett. 10:1261-4 (2000), Ottosen et al., J. Med. Chem. 46:5651-62 (2003); Thurmond et al., Eur. J. Biochem. 268:5747-54 (2001), Jackson et al., Curr. Top. Med. Chem. 2:1011-20 (2002); Jeohn et al., Neuroscience 114:689-97 (2002); Revesz et al., Bioorg. Med. Chem. Lett. 12:2109-12 (2002); Orchard, Curr. Opin. Drug Discov. Devel. 5:713-7 (2002); Nishikori et al., Eur. J. Pharmacol. 451:327-33 (2002); Foster et al., Drug News Perspect. 13:488-97 (2000); Boehm et al., Bioorg. Med. Chem. Lett. 11:1123-6 (2001); Hunt et al., Bioorg. Med. Chem. Lett. 13:467-70 (2003); de Laszlo et al., Bioorg. Med. Chem. Lett. 8:2689-94 (1998); McIntyre et al., Bioorg. Med. Chem. Lett. 12:689-92 (2002); Haddad et al., Curr. Opin. Investig. Drugs. 2:1070-6 (2002); Collis et al., Bioorg. Med. Chem. Lett. 11:693-6 (20001).

JNK Kinase Inhibitors

JNK Kinase inhibitors are described, for example, in Bogoyevitch et al., Biochim. Biophys. Acta. 1697:89-101 (2004) and in U.S. Patent Application Publication Nos. 20040092562, 20040087642, 20040087615, 20040082509, 20040077877, 20040072888, 20040063946, 20040023963, 20030220330, 20030162794, 20030153560, 20030108539, 20030100549, 20030096816, 20030087922, 2003073732, 20020111353, 20020103229, 20020119135, and 20040077632.

Other Therapeutic Agents

If desired, the combination of the invention containing the agent that increases signaling activity of the glucocorticoid receptor and a non-steroidal agent that modulates the signaling activity of one or more of the NFκB, NFAT, Elk-1, or AP-1 signaling pathways such that proinflammatory cytokine secretion or production or any other inflammatory response is reduced may be formulated or administered with additional therapeutic agents. Such agents include, for example, corticosteroids, NSAID, COX-2 inhibitor, DMARD, biologic, xanthine, anticholinergic compound, beta receptor agonist, bronchodilator, non-steroidal calcineurin inhibitor, vitamin D analog, psoralen, retinoid, and 5-amino salicylic acid.

Corticosteroids

Optionally, a corticosteroid may be formulated in the composition of the invention or administered to the mamamal being treated according to the invention. Suitable corticosteroids include 11-alpha, 17-alpha,21-trihydroxypregn-4-ene-3,20-dione; 11-beta, 16-alpha,17,21-tetrahydroxypregn-4-ene-3,20-dione; 11-beta,16-alpha,17,21-tetrahydroxypregn-1,4-diene-3,20-dione; 11-beta,17-alpha,21-trihydroxy-6-alpha-methylpregn-4-ene-3,20-dione; 11-dehydrocorticosterone; 11-deoxycortisol; 11-hydroxy-1,4-androstadiene-3,17-dione; 11-ketotestosterone; 14-hydroxyandrost-4-ene-3,6,17-trione; 15,17-dihydroxyprogesterone; 16-methylhydrocortisone; 17,21-dihydroxy-16-alpha-methylpregna-1,4,9(11)-triene-3,20-dione; 17-alpha-hydroxypregn-4-ene-3,20-dione; 17-alpha-hydroxypregnenolone; 17-hydroxy-16-beta-methyl-5-beta-pregn-9(11)-ene-3,20-dione; 17-hydroxy-4,6,8(14)-pregnatriene-3,20-dione; 17-hydroxypregna-4,9(11)-diene-3,20-dione; 18-hydroxycorticosterone; 18-hydroxycortisone; 18-oxocortisol; 21-deoxyaldosterone; 21-deoxycortisone; 2-deoxyecdysone; 2-methylcortisone; 3-dehydroecdysone; 4-pregnene-17-alpha,20-beta, 21-triol-3,11-dione; 6,17,20-trihydroxypregn-4-ene-3-one; 6-alpha-hydroxycortisol; 6-alpha-fluoroprednisolone, 6-alpha-methylprednisolone, 6-alpha-methylprednisolone 21-acetate, 6-alpha-methylprednisolone 21-hemisuccinate sodium salt, 6-beta-hydroxycortisol, 6-alpha, 9-alpha-difluoroprednisolone 21-acetate 17-butyrate, 6-hydroxycorticosterone; 6-hydroxydexamethasone; 6-hydroxyprednisolone; 9-fluorocortisone; alclometasone dipropionate; aldosterone; algestone; alphaderm; amadinone; amcinonide; anagestone; androstenedione; anecortave acetate; beclomethasone; beclomethasone dipropionate; beclomethasone dipropionate monohydrate; betamethasone 17-valerate; betamethasone sodium acetate; betamethasone sodium phosphate; betamethasone valerate; bolasterone; budesonide; calusterone; chlormadinone; chloroprednisone; chloroprednisone acetate; cholesterol; clobetasol; clobetasol propionate; clobetasone; clocortolone; clocortolone pivalate; clogestone; cloprednol; corticosterone; cortisol; cortisol acetate; cortisol butyrate; cortisol cypionate; cortisol octanoate; cortisol sodium phosphate; cortisol sodium succinate; cortisol valerate; cortisone; cortisone acetate; cortodoxone; daturaolone; deflazacort, 21-deoxycortisol, dehydroepiandrosterone; delmadinone; deoxycorticosterone; deprodone; descinolone; desonide; desoximethasone; dexafen; dexamethasone; dexamethasone 21-acetate; dexamethasone acetate; dexamethasone sodium phosphate; dichlorisone; diflorasone; diflorasone diacetate; diflucortolone; dihydroelatericin a; domoprednate; doxibetasol; ecdysone; ecdysterone; endrysone; enoxolone; flucinolone; fludrocortisone; fludrocortisone acetate; flugestone; flumethasone; flumethasone pivalate; flumoxonide; flunisolide; fluocinolone; fluocinolone acetonide; fluocinonide; 9-fluorocortisone; fluocortolone; fluorohydroxyandrostenedione; fluorometholone; fluorometholone acetate; fluoxymesterone; fluprednidene; fluprednisolone; flurandrenolide; fluticasone; fluticasone propionate; formebolone; formestane; formocortal; gestonorone; glyderinine; halcinonide; hyrcanoside; halometasone; halopredone; haloprogesterone; hydrocortiosone cypionate; hydrocortisone; hydrocortisone 21-butyrate; hydrocortisone aceponate; hydrocortisone acetate; hydrocortisone buteprate; hydrocortisone butyrate; hydrocortisone cypionate; hydrocortisone hemisuccinate; hydrocortisone probutate; hydrocortisone sodium phosphate; hydrocortisone sodium succinate; hydrocortisone valerate; hydroxyprogesterone; inokosterone; isoflupredone; isoflupredone acetate; isoprednidene; meclorisone; mecortolon; medrogestone; medroxyprogesterone; medrysone; megestrol; megestrol acetate; melengestrol; meprednisone; methandrostenolone; methylprednisolone; methylprednisolone aceponate; methylprednisolone acetate; methylprednisolone hemisuccinate; methylprednisolone sodium succinate; methyltestosterone; metribolone; mometasone; mometasone furoate; mometasone furoate monohydrate; nisone; nomegestrol; norgestomet; norvinisterone; oxymesterone; paramethasone; paramethasone acetate; ponasterone; prednisolamate; prednisolone; prednisolone 21-hemisuccinate; prednisolone acetate; prednisolone farnesylate; prednisolone hemisuccinate; prednisolone-21(beta-D-glucuronide); prednisolone metasulphobenzoate; prednisolone sodium phosphate; prednisolone steaglate; prednisolone tebutate; prednisolone tetrahydrophthalate; prednisone; prednival; prednylidene; pregnenolone; procinonide; tralonide; progesterone; promegestone; rhapontisterone; rimexolone; roxibolone; rubrosterone; stizophyllin; tixocortol; topterone; triamcinolone; triamcinolone acetonide; triamcinolone acetonide 21-palmitate; triamcinolone diacetate; triamcinolone hexacetonide; trimegestone; turkesterone; and wortmannin.

Standard recommended dosages for various steroid/disease combinations are provided in Table 1, below.

TABLE 1
Standard recommended corticosteroid dosages

| Indication | Route | Drug | Dose | Schedule |
|---|---|---|---|---|
| Psoriasis | oral | prednisolone | 7.5-60 mg | per day or divided b.i.d. |
| | oral | prednisone | 7.5-60 mg | per day or divided b.i.d. |
| Asthma | inhaled | beclomethasone dipropionate | 42 μg/puff) | 4-8 puffs b.i.d. |
| | inhaled | budesonide | (200 μg/inhalation) | 1-2 inhalations b.i.d. |
| | inhaled | flunisolide | (250 μg/puff) | 2-4 puffs b.i.d. |
| | inhaled | fluticasone propionate | (44, 110 or 220 μg/puff) | 2-4 puffs b.i.d. |
| | inhaled | triamcinolone acetonide | (100 μg/puff) | 2-4 puffs b.i.d. |
| COPD | oral | prednisone | 30-40 mg | per day |
| Crohn's disease | oral | budesonide | 9 mg | per day |
| Ulcerative colitis | oral | prednisone | 40-60 mg | per day |
| | oral | hydrocortisone | 300 mg (IV) | per day |
| | oral | methylprednisolone | 40-60 mg | per day |
| Rheumatoid arthritis | oral | prednisone | 7.5-10 mg | per day |

Other standard recommended dosages for corticosteroids are provided, e.g., in the Merck Manual of Diagnosis & Therapy (17th Ed. MH Beers et al., Merck & Co.) and Physicians' Desk Reference 2003 (57^{th} Ed. Medical Economics Staff et al., Medical Economics Co., 2002). In one embodiment, the dosage of corticosteroid administered is a dosage equivalent to a prednisolone dosage, as defined herein. For example, a low dosage of a corticosteroid may be considered as the dosage equivalent to a low dosage of prednisolone.

Other compounds that may be used as a substitute for or in addition to a corticosteroid in the methods, compositions, and kits of the invention A-348441 (Karo Bio), adrenal cortex extract (GlaxoSmithKline), alsactide (Aventis), amebucort (Schering AG), amelometasone (Taisho), ATSA (Pfizer), bitolterol (Elan), CBP-2011 (InKine Pharmaceutical), cebaracetam (Novartis) CGP-13774 (Kissei), ciclesonide (Altana), ciclometasone (Aventis), clobetasone butyrate (GlaxoSmithKline), cloprednol (Hoffmann-La Roche), collismycin A (Kirin), cucurbitacin E (NIH), deflazacort (Aventis), deprodone propionate (SSP), dexamethasone acefurate (Schering-Plough), dexamethasone linoleate (GlaxoSmithKline), dexamethasone valerate (Abbott), difluprednate (Pfizer), domoprednate (Hoffmann-La Roche), ebiratide (Aventis), etiprednol dicloacetate (IVAX), fluazacort (Vicuron), flumoxonide (Hoffmann-La Roche), fluocortin butyl (Schering AG), fluocortolone monohydrate (Schering AG), GR-250495X (GlaxoSmithKline), halometasone (Novartis), halopredone (Dainippon), HYC-141 (Fidia), icomethasone enbutate (Hovione), itrocinonide (AstraZeneca), L-6485 (Vicuron), Lipocort (Draxis Health), locicortone (Aventis), meclorisone (Schering-Plough), naflocort (Bristol-Myers Squibb), NCX-1015 (NicOx), NCX-1020 (NicOx), NCX-1022 (NicOx), nicocortonide (Yamanouchi), NIK-236 (Nikken Chemicals), NS-126 (SSP), Org-2766 (Akzo Nobel), Org-6632 (Akzo Nobel), P16CM, propylmesterolone (Schering AG), RGH-1113 (Gedeon Richter), rofleponide (AstraZeneca), rofleponide palmitate (AstraZeneca), RPR-106541 (Aventis), RU-26559 (Aventis), Sch-19457 (Schering-Plough), T25 (Matrix Therapeutics), TBI-PAB (Sigma-Tau), ticabesone propionate (Hoffmann-La Roche), tifluadom (Solvay), timobesone (Hoffmann-La Roche), TSC-5 (Takeda), and ZK-73634 (Schering AG).

Disease-Specific Therapeutic Agents

Chronic Obstructive Pulmonary Disease

In one embodiment, the methods, compositions, and kits of the invention are used for the treatment of chronic obstructive pulmonary disease (COPD). If desired, one or more agents typically used to treat COPD may be used as a substitute for or in addition to the combination in the methods, compositions, and kits of the invention. Such agents include xanthines (e.g., theophylline), anticholinergic compounds (e.g., ipratropium, tiotropium), biologics, small molecule immunomodulators, and beta receptor agonists/bronchdilators (e.g., Ibuterol sulfate, bitolterol mesylate, epinephrine, formoterol fumarate, isoproteronol, levalbuterol hydrochloride, metaproterenol sulfate, pirbuterol scetate, salmeterol xinafoate, and terbutaline).

Psoriasis

The methods, compositions, and kits of the invention may be used for the treatment of psoriasis. If desired, one or more antipsoriatic agents typically used to treat psoriasis may be used as a substitute for or in addition to the combination the invention. Such agents include biologics (e.g., alefacept, inflixamab, adelimumab, efalizumab, etanercept, and CDP-870), small molecule immunomodulators (e.g., VX 702, SCIO 469, doramapimod, RO 30201195, SCIO 323, DPC 333, pranalcasan, mycophenolate, and merimepodib), non-steroidal calcineurin inhibitors (e.g., cyclosporine, tacrolimus, pimecrolimus, and ISAtx247), vitamin D analogs (e.g., calcipotriene, calcipotriol), psoralens (e.g., methoxsalen), retinoids (e.g., acitretin, tazoretene), DMARDs (e.g., methotrexate), and anthralin. Thus, in one embodiment, the invention features the combination of an agent that increases the signaling activity of a glucocorticoid receptor, a non-steroidal agent that reduces the signaling activity of one or more of the NFκB, NFAT, AP-1, Elk-1 signaling pathways, and an antipsoriatic agent, and methods of treating psoriasis therewith.

Inflammatory Bowel Disease

The methods, compositions, and kits of the invention may be used for the treatment of inflammatory bowel disease. If desired, one or more agents typically used to treat inflammatory bowel disease may be used in addition to the combination featured in the methods, compositions, and kits of the invention. Such agents include biologics (e.g., inflixamab, adelimumab, and CDP-870), small molecule immunomodulators (e.g., VX 702, SCIO 469, doramapimod, RO 30201195, SCIO 323, DPC 333, pranalcasan, mycophenolate, and merimepodib), non-steroidal calcineurin inhibitors (e.g., cyclosporine, tacrolimus, pimecrolimus, and ISAtx247), 5-amino salicylic acid (e.g., mesalamine, sulfasalazine, balsalazide disodium, and olsalazine sodium), DMARDs (e.g., methotrexate and azathioprine) and alosetron. Thus, in one embodiment, the invention features the combination of an agent that increases the signaling activity of a glucocorticoid receptor, a non-steroidal agent that reduces the signaling activity of one or more of the NFκB, NFAT, AP-1, Elk-1 signaling pathways, and any of the foregoing agents, and methods of treating inflammatory bowel disease therewith.

Rheumatoid Arthritis

The methods, compositions, and kits of the invention may be used for the treatment of rheumatoid arthritis. If desired, one or more agents typically used to treat rheumatoid arthritis may be used in addition to the combination featured in the methods, compositions, and kits of the invention. Such agents include NSAIDs (e.g., naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid (salsalate), fenoprofen, flurbiprofen, ketoprofen, meclofenamate sodium, meloxicam, oxaprozin, sulindac, and tolmetin), COX-2 inhibitors (e.g., rofecoxib, celecoxib, valdecoxib, and lumiracoxib), biologics (e.g., inflixamab, adelimumab, etanercept, CDP-870, rituximab, and atlizumab), small molecule immunomodulators (e.g., VX 702, SCIO 469, doramapimod, RO 30201195, SCIO 323, DPC 333, pranalcasan, mycophenolate, and merimepodib), non-steroidal calcineurin inhibitors (e.g., cyclosporine, tacrolimus, pimecrolimus, and ISAtx247), 5-amino salicylic acid (e.g., mesalamine, sulfasalazine, balsalazide disodium, and olsalazine sodium), DMARDs (e.g., methotrexate, leflunomide, minocycline, auranofin, gold sodium thiomalate, aurothioglucose, and azathioprine), hydroxychloroquine sulfate, and penicillamine. Thus, in one embodiment, the invention features the combination of an agent that increases the signaling activity of a glucocorticoid receptor, a non-steroidal agent that reduces the signaling activity of one or more of the NFκB, NFAT, AP-1, Elk-1 signaling pathways, with any of the foregoing agents, and methods of treating rheumatoid arthritis therewith.

Asthma

The methods, compositions, and kits of the invention may be used for the treatment of asthma. If desired, one or more agents typically used to treat asthma may be used in addition to a corticosteroid in the methods, compositions, and kits of the invention. Such agents include beta 2 agonists/bronchodilators/leukotriene modifiers (e.g., zafirlukast, montelukast, and zileuton), biologics (e.g., omalizumab), small molecule immunomodulators, anticholinergic compounds, xanthines, ephedrine, guaifenesin, cromolyn sodium, nedocromil sodium, and potassium iodide. Thus, in one embodiment, the invention features the combination of an agent that increases the signaling activity of a glucocorticoid receptor, a non-steroidal agent that reduces the signaling activity of one or more of the NFκB, NFAT, AP-1, Elk-1 signaling pathways and any of the foregoing agents, and methods of treating rheumatoid arthritis therewith.

Non-Steroidal Immunophilin-Dependent Immunosuppressants

In one embodiment, the invention features methods, compositions, and kits employing an agent that increases the signaling activity of a glucocorticoid receptor, a non-steroidal agent that reduces the signaling activity of one or more of the NFκB, NFAT, AP-1, Elk-1 signaling pathways, and a non-steroidal immunophilin-dependent immunosuppressant (NsIDI).

In healthy individuals the immune system uses cellular effectors, such as B-cells and T-cells, to target infectious microbes and abnormal cell types while leaving normal cells intact. In individuals with an autoimmune disorder or a transplanted organ, activated T-cells damage healthy tissues. Calcineurin inhibitors (e.g., cyclosporines, tacrolimus, pimecrolimus), and rapamycin target many types of immunoregulatory cells, including T-cells, and suppress the immune response in organ transplantation and autoimmune disorders.

Cyclosporines

The cyclosporines are fungal metabolites that comprise a class of cyclic oligopeptides that act as immunosuppressants. Cyclosporine A, and its deuterated analogue ISAtx247, is a hydrophobic cyclic polypeptide consisting of eleven amino acids. Cyclosporine A binds and forms a complex with the intracellular receptor cyclophilin. The cyclosporine/cyclophilin complex binds to and inhibits calcineurin, a Ca²⁺-calmodulin-dependent serine-threonine-specific protein phosphatase. Calcineurin mediates signal transduction events required for T-cell activation (reviewed in Schreiber et al., Cell 70:365-368, 1991). Cyclosporines and their functional and structural analogs suppress the T-cell-dependent immune response by inhibiting antigen-triggered signal transduction. This inhibition decreases the expression of proinflammatory cytokines, such as IL-2.

Many cyclosporines (e.g., cyclosporine A, B, C, D, E, F, G, H, and I) are produced by fungi. Cyclosporine A is a commercially available under the trade name NEORAL from Novartis. Cyclosporine A structural and functional analogs include cyclosporines having one or more fluorinated amino acids (described, e.g., in U.S. Pat. No. 5,227,467); cyclosporines having modified amino acids (described, e.g., in U.S. Pat. Nos. 5,122,511 and 4,798,823); and deuterated cyclosporines, such as ISAtx247 (described in U.S. Patent Publication No. 20020132763). Additional cyclosporine analogs are described in U.S. Pat. Nos. 6,136,357, 4,384,996, 5,284,826, and 5,709,797. Cyclosporine analogs include, but are not limited to, D-Sar (α-SMe)³ Val²-DH-Cs (209-825), Allo-Thr-2-Cs, Norvaline-2-Cs, D-Ala (3-acetylamino)-8-Cs, Thr-2-Cs, and D-MeSer-3-Cs, D-Ser (O—CH₂CH₂—OH)-8-Cs, and D-Ser-8-Cs, which are described in Cruz et al. (Antimicrob. Agents Chemother. 44:143-149, 2000).

Cyclosporines are highly hydrophobic and readily precipitate in the presence of water (e.g., on contact with body fluids). Methods of providing cyclosporine formulations with improved bioavailability are described in U.S. Pat. Nos. 4,388,307, 6,468,968, 5,051,402, 5,342,625, 5,977,066, and 6,022,852. Cyclosporine microemulsion compositions are described in U.S. Pat. Nos. 5,866,159, 5,916,589, 5,962,014, 5,962,017, 6,007,840, and 6,024,978.

Cyclosporines can be administered either intravenously or orally, but oral administration is preferred. To counteract the hydrophobicity of cyclosporine A, an intravenous cyclosporine A is usually provided in an ethanol-polyoxyethylated castor oil vehicle that must be diluted prior to administration. Cyclosporine A may be provided, e.g., as a microemulsion in a 25 mg or 100 mg tablets, or in a 100 mg/ml oral solution (NEORAL™).

Typically, patient dosage of an oral cyclosporine varies according to the patient's condition, but some standard recommended dosages in prior art treatment regimens are provided herein. Patients undergoing organ transplant typically receive an initial dose of oral cyclosporine A in amounts between 12 and 15 mg/kg/day. Dosage is then gradually decreased by 5% per week until a 7-12 mg/kg/day maintenance dose is reached. For intravenous administration 2-6 mg/kg/day is preferred for most patients. For patients diagnosed as having Crohn's disease or ulcerative colitis, dosage amounts from 6-8 mg/kg/day are generally given. For patients diagnosed as having systemic lupus erythematosus, dosage amounts from 2.2-6.0 mg/kg/day are generally given. For psoriasis or rheumatoid arthritis, dosage amounts from 0.5-4 mg/kg/day are typical. Other useful dosages include 0.5-5 mg/kg/day, 5-10 mg/kg/day, 10-15 mg/kg/day, 15-20 mg/kg/day, or 20-25 mg/kg/day. Often cyclosporines are administered in combination with other immunosuppressive agents, such as glucocorticoids. Additional information is provided in Table 2.

TABLE 2
NsIDIs

| | Atopic | | | | | | |
|---|---|---|---|---|---|---|---|
| Compound | Dermatitis | Psoriasis | RA | Crohn's | UC | Transplant | SLE |
| CsA | N/A | 0.5-4 mg/kg/day | 0.5-4 mg/kg/day | 6-8 mg/kg/day | 6-8 mg/kg/day | ˜7-12 | 2.2-6.0 |
| (NEORAL) | | | | (oral- | (oral) | mg/kg/day | mg/kg/day |
| | | | | fistulizing) | | | |
| Tacrolimus | .03-0.1% | .05-1.15 mg/kg/day | 1-3 mg/day | 0.1-0.2 mg/kg/day | 0.1-0.2 mg/kg/day | 0.1-0.2 | N/A |
| | cream/twice | (oral) | (oral) | (oral) | (oral) | mg/kg/day | |
| | day (30 and | | | | | (oral) | |
| | 60 gram | | | | | | |
| | tubes) | | | | | | |
| Pimecrolimus | 1% | 40-60 mg/day | 40-60 mg/day | 80-160 mg/day | 160-240 mg/day | 40-120 | 40-120 |
| | cream/twice | (oral) | (oral) | (oral) | (oral) | mg/day | mg/day |
| | day (15, 30, | | | | | (oral) | (oral) |
| | 100 gram | | | | | | |
| | tubes) | | | | | | |

Legend
CsA = cyclosporine A
RA = rheumatoid arthritis
UC = ulcerative colitis
SLE = systemic lupus erythamatosus

Tacrolimus

Tacrolimus (PROGRAF, Fujisawa), also known as FK506, is an immunosuppressive agent that targets T-cell intracellular signal transduction pathways. Tacrolimus binds to an intracellular protein FK506 binding protein (FKBP-12) that is not structurally related to cyclophilin (Harding et al. Nature 341:758-7601, 1989; Siekienka et al. Nature 341:755-757, 1989; and Soltoff et al., J. Biol. Chem. 267:17472-17477, 1992). The FKBP/FK506 complex binds to calcineurin and inhibits calcineurin's phosphatase activity. This inhibition prevents the dephosphorylation and nuclear translocation of NFAT, a nuclear component that initiates gene transcription required for lymphokine (e.g., IL-2, gamma interferon) production and T-cell activation. Thus, tacrolimus inhibits T-cell activation.

Tacrolimus is a macrolide antibiotic that is produced by Streptomyces tsukubaensis. It suppresses the immune system and prolongs the survival of transplanted organs. It is currently available in oral and injectable formulations. Tacrolimus capsules contain 0.5 mg, 1 mg, or 5 mg of anhydrous tacrolimus within a gelatin capsule shell. The injectable formulation contains 5 mg anhydrous tacrolimus in castor oil and alcohol that is diluted with 9% sodium chloride or 5% dextrose prior to injection. While oral administration is preferred, patients unable to take oral capsules may receive injectable tacrolimus. The initial dose should be administered no sooner than six hours after transplant by continuous intravenous infusion.

Tacrolimus and tacrolimus analogs are described by Tanaka et al., (J. Am. Chem. Soc., 109:5031, 1987), and in U.S. Pat. Nos. 4,894,366, 4,929,611, and 4,956,352. FK506-related compounds, including FR-900520, FR-900523, and FR-900525, are described in U.S. Pat. No. 5,254,562; O-aryl, O-alkyl, O-alkenyl, and O-alkynylmacrolides are described in U.S. Pat. Nos. 5,250,678, 532,248, 5,693,648; amino O-aryl macrolides are described in U.S. Pat. No. 5,262,533; alkylidene macrolides are described in U.S. Pat. No. 5,284,840; N-heteroaryl, N-alkylheteroaryl, N-alkenylheteroaryl, and N-alkynylheteroaryl macrolides are described in U.S. Pat. No. 5,208,241; aminomacrolides and derivatives thereof are described in U.S. Pat. No. 5,208,228; fluoromacrolides are described in U.S. Pat. No. 5,189,042; amino O-alkyl, O-alkenyl, and O-alkynylmacrolides are described in U.S. Pat. No. 5,162,334; and halomacrolides are described in U.S. Pat. No. 5,143,918.

While suggested dosages will vary with a patient's condition, standard recommended dosages used in prior art treatment regimens are provided below. Patients diagnosed as having Crohn's disease or ulcerative colitis are administered 0.1-0.2 mg/kg/day oral tacrolimus. Patients having a transplanted organ typically receive doses of 0.1-0.2 mg/kg/day of oral tacrolimus. Patients being treated for rheumatoid arthritis typically receive 1-3 mg/day oral tacrolimus. For the treatment of psoriasis, 0.01-0.15 mg/kg/day of oral tacrolimus is administered to a patient. Atopic dermatitis can be treated twice a day by applying a cream having 0.03-0.1% tacrolimus to the affected area. Patients receiving oral tacrolimus capsules typically receive the first dose no sooner than six hours after transplant, or eight to twelve hours after intravenous tacrolimus infusion was discontinued. Other suggested tacrolimus dosages include 0.005-0.01 mg/kg/day, 0.01-0.03 mg/kg/day, 0.03-0.05 mg/kg/day, 0.05-0.07 mg/kg/day, 0.07-0.10 mg/kg/day, 0.10-0.25 mg/kg/day, or 0.25-0.5 mg/kg/day.

Tacrolimus is extensively metabolized by the mixed-function oxidase system, in particular, by the cytochrome P-450 system. The primary mechanism of metabolism is demethylation and hydroxylation. While various tacrolimus metabolites are likely to exhibit immunosuppressive biological activity, the 13-demethyl metabolite is reported to have the same activity as tacrolimus.

Pimecrolimus and Ascomycin Derivatives

Ascomycin is a close structural analog of FK506 and is a potent immunosuppressant. It binds to FKBP-12 and suppresses its proline rotamase activity. The ascomycin-FKBP complex inhibits calcineurin, a type 2B phosphatase.

Pimecrolimus (also known as SDZ ASM-981) is an 33-epi-chloro derivative of the ascomycin. It is produced by the strain Streptomyces hygroscopicus var. ascomyceitus. Like tacrolimus, pimecrolimus (ELIDEL™, Novartis) binds FKBP-12, inhibits calcineurin phosphatase activity, and inhibits T-cell activation by blocking the transcription of early cytokines. In particular, pimecrolimus inhibits IL-2 production and the release of other proinflammatory cytokines.

Pimecrolimus structural and functional analogs are described in U.S. Pat. No. 6,384,073. Pimecrolimus is particularly useful for the treatment of atopic dermatitis. Pimecrolimus is currently available as a 1% cream. While individual dosing will vary with the patient's condition, some standard recommended dosages are provided below. Oral pimecrolimus can be given for the treatment of psoriasis or rheumatoid arthritis in amounts of 40-60 mg/day. For the treatment of Crohn's disease or ulcerative colitis amounts of 80-160 mg/day pimecrolimus can be given. Patients having an organ transplant can be administered 160-240 mg/day of pimecrolimus. Patients diagnosed as having systemic lupus erythamatosus can be administered 40-120 mg/day of pimecrolimus. Other useful dosages of pimecrolimus include 0.5-5 mg/day, 5-10 mg/day, 10-30 mg/day, 40-80 mg/day, 80-120 mg/day, or even 120-200 mg/day.

Rapamycin

Rapamycin (Rapamune® sirolimus, Wyeth) is a cyclic lactone produced by Steptomyces hygroscopicus. Rapamycin is an immunosuppressive agent that inhibits T-lymphocyte activation and proliferation. Like cyclosporines, tacrolimus, and pimecrolimus, rapamycin forms a complex with the immunophilin FKBP-12, but the rapamycin-FKBP-12 complex does not inhibit calcineurin phosphatase activity. The rapamycin-immunophilin complex binds to and inhibits the mammalian target of rapamycin (mTOR), a kinase that is required for cell cycle progression. Inhibition of mTOR kinase activity blocks T-lymphocyte proliferation and lymphokine secretion.

Rapamycin structural and functional analogs include mono- and diacylated rapamycin derivatives (U.S. Pat. No. 4,316,885); rapamycin water-soluble prodrugs (U.S. Pat. No. 4,650,803); carboxylic acid esters (PCT Publication No. WO 92/05179); carbamates (U.S. Pat. No. 5,118,678); amide esters (U.S. Pat. No. 5,118,678); biotin esters (U.S. Pat. No. 5,504,091); fluorinated esters (U.S. Pat. No. 5,100,883); acetals (U.S. Pat. No. 5,151,413); silyl ethers (U.S. Pat. No. 5,120,842); bicyclic derivatives (U.S. Pat. No. 5,120,725); rapamycin dimers (U.S. Pat. No. 5,120,727); O-aryl, O-alkyl, O-alkyenyl and O-alkynyl derivatives (U.S. Pat. No. 5,258,389); and deuterated rapamycin (U.S. Pat. No. 6,503,921). Additional rapamycin analogs are described in U.S. Pat. Nos. 5,202,332 and 5,169,851.

Everolimus (40-O-(2-hydroxyethyl)rapamycin; CERTICAN™; Novartis) is an immunosuppressive macrolide that is structurally related to rapamycin, and has been found to be particularly effective at preventing acute rejection of organ transplant when give in combination with cyclosporin A.

Rapamycin is currently available for oral administration in liquid and tablet formulations. RAPAMUNE™ liquid contains 1 mg/mL rapamycin that is diluted in water or orange juice prior to administration. Tablets containing 1 or 2 mg of rapamycin are also available. Rapamycin is preferably given once daily as soon as possible after transplantation. It is absorbed rapidly and completely after oral administration. Typically, patient dosage of rapamycin varies according to the patient's condition, but some standard recommended dosages are provided below. The initial loading dose for rapamycin is 6 mg. Subsequent maintenance doses of 2 mg/day are typical. Alternatively, a loading dose of 3 mg, 5 mg, 10 mg, 15 mg, 20 mg, or 25 mg can be used with a 1 mg, 3 mg, 5 mg, 7 mg, or 10 mg per day maintenance dose. In patients weighing less than 40 kg, rapamycin dosages are typically adjusted based on body surface area; generally a 3 mg/m²/day loading dose and a 1-mg/m²/day maintenance dose is used.

Peptide Moieties

Peptides, peptide mimetics, peptide fragments, either natural, synthetic or chemically modified, that impair the NFAT, NFκB, AP-1, or Elk-1 signaling pathway are suitable for use in practicing the invention. Examples of peptides that act as calcineurin inhibitors by inhibiting the NFAT activation and the NFAT transcription factor are described, e.g., by Aramburu et al., Science 285:2129-2133, 1999) and Aramburu et al., Mol. Cell 1:627-637, 1998). As a class of calcinuerin inhibitors, these agents are useful in the methods of the invention.

Exemplary inhibitors include compounds that reduce the amount of target protein or RNA levels (e.g., antisense compounds, dsRNA, ribozymes) and compounds that compete with endogenous mitotic kinesins or protein tyrosine phosphatases for binding partners (e.g., dominant negative proteins or polynucleotides encoding the same).

Antisense Compounds

The biological activity of a mitotic kinesin and/or protein tyrosine phosphatase can be reduced through the use of an antisense compound directed to RNA encoding the target protein. Antisense compounds that reduce expression of signaling molecules can be identified using standard techniques. For example, accessible regions of the target the mRNA of the signaling molecule can be predicted using an RNA secondary structure folding program such as MFOLD (M. Zuker, D. H. Mathews & D. H. Turner, Algorithms and Thermodynamics for RNA Secondary Structure Prediction: A Practical Guide. In: RNA Biochemistry and Biotechnology, J. Barciszewski & B. F. C. Clark, eds., NATO ASI Series, Kluwer Academic Publishers, (1999)). Sub-optimal folds with a free energy value within 5% of the predicted most stable fold of the mRNA are predicted using a window of 200 bases within which a residue can find a complimentary base to form a base pair bond. Open regions that do not form a base pair are summed together with each suboptimal fold and areas that are predicted as open are considered more accessible to the binding to antisense nucleobase oligomers. Other methods for antisense design are described, for example, in U.S. Pat. No. 6,472,521, Antisense Nucleic Acid Drug Dev. 1997 7:439-444, Nucleic Acids Research 28:2597-2604, 2000, and Nucleic Acids Research 31:4989-4994, 2003.

RNA Interference

The biological activity of a signaling molecule can be reduced through the use of RNA interference (RNAi), employing, e.g., a double stranded RNA (dsRNA) or small interfering RNA (siRNA) directed to the signaling molecule in question (see, e.g., Miyamoto et al., Prog. Cell Cycle Res. 5:349-360, 2003; U.S. Patent Application Publication No. 20030157030). Methods for designing such interfering RNAs are known in the art. For example, software for designing interfering RNA is available from Oligoengine (Seattle, Wash.).

Dominant Negative Proteins

One skilled in the art would know how to make dominant negative proteins to the signaling molecules to be targeted. Such dominant negative proteins are described, for example, in Gupta et al., J. Exp. Med., 186:473-478, 1997; Maegawa et al., J. Biol. Chem. 274:30236-30243, 1999; Woodford-Thomas et al., J. Cell Biol. 117:401-414, 1992;

Assays for Proinflammatory Cytokine-Suppressing Activity

The therapeutic or anti-inflammatory efficacy of the combinations of the invention may be determined by any standard method known in the art or as described herein. For example, the expression level or the biological activity of any of the signaling molecule involved in the targeted signaling pathway may be determined by any standard method known in the art (e.g., phosphorylation studies, western and northern analysis, ELISA, and immunohistochemistry). If the expression or biological activity of the signaling molecule is reduced relative to such expression or biological activity in an untreated control, the combination is identified as being useful according to the invention. In this case, the signaling molecule has a role downstream of the point in the signaling pathway is targeted. If desired, the expression level or biological activity of NFκB, NFAT, AP-1, and Elk-1 may also be determined.

In addition to detecting the expression level or biological activity of signaling molecules in the signaling pathway, the anti-inflammatory efficacy of the combinations of the invention may be determined by assaying for the release or production of pro-inflammatory cytokines (as described herein). TNF-α production may be assessed, for example, by measuring TNF-α transcription or by measuring TNF-α protein levels by ELISA. Compound dilution matrices may be assayed for the suppression of TNFα, IFNγ, IL-1β, IL-2, IL-4, and IL-5 as described below.

TNFα

A 100 μl suspension of diluted human white blood cells contained within each well of a polystyrene 384-well plate (NalgeNunc) is stimulated to secrete TNFα by treatment with a final concentration of 2 μg/mL lipopolysaccharide (Sigma L-4130). Various concentrations of each test compound are added at the time of stimulation. After 16-18 hours of incubation at 37° C. in a humidified incubator, the plate is centrifuged and the supernatant transferred to a white opaque polystyrene 384 well plate (NalgeNunc, Maxisorb) coated with an anti-TNFα antibody (PharMingen, #551220). After a two-hour incubation, the plate is washed (Tecan PowerWasher 384) with PBS containing 0.1% Tween 20 and incubated for an additional one hour with another anti-TNFα antibody that was biotin labeled (PharMingen, #554511) and HRP coupled to strepavidin (PharMingen, #13047E). After the plate is washed with 0.1% Tween 20/PBS, an HRP-luminescent substrate is added to each well and light intensity measured using a LJL Analyst plate luminometer.

IFNγ

A 100 μL suspension of diluted human white blood cells contained within each well of a polystyrene 384-well plate (NalgeNunc) is stimulated to secrete IFNγ by treatment with a final concentration of 10 ng/mL phorbol 12-myristate 13-acetate (Sigma, P-1585) and 750 ng/mL ionomycin (Sigma, 1-0634). Various concentrations of each test compound are added at the time of stimulation. After 16-18 hours of incubation at 37° C. in a humidified incubator, the plate is centrifuged and the supernatant transferred to a white opaque polystyrene 384 well plate (NalgeNunc, Maxisorb) coated with an anti-IFNγ antibody (Endogen, #M-700A-E). After a two-hour incubation, the plate is washed (Tecan PowerWasher 384) with phosphate buffered saline (PBS) containing 0.1% Tween 20 (polyoxyethylene sorbitan monolaurate) and incubated for an additional one hour with another anti-IFNγ antibody that was biotin labeled (Endogen, M701B) and horseradish peroxidase (HRP) coupled to strepavidin (PharMingen, #13047E). After the plate is washed with 0.1% Tween 20/PBS, an HRP-luminescent substrate is added to each well and light intensity measured using a LJL Analyst plate luminometer.

IL-1β

A 100 μL suspension of diluted human white blood cells contained within each well of a polystyrene 384-well plate (NalgeNunc) is stimulated to secrete IL-1β by treatment with a final concentration of 2 μg/mL lipopolysaccharide (Sigma L-4130). Various concentrations of each test compound are added at the time of stimulation. After 16-18 hours of incubation at 37° C. in a humidified incubator, the plate is centrifuged and the supernatant transferred to a white opaque polystyrene 384 well plate (NalgeNunc, Maxisorb) coated with an anti-IL-1, antibody (R&D, #MAB-601). After a two-hour incubation, the plate is washed (Tecan PowerWasher 384) with PBS containing 0.1% Tween 20 and incubated for an additional one hour with another anti-IL-1β antibody that is biotin labeled (R&D, BAF-201) and HRP coupled to strepavidin (PharMingen, #13047E). After the plate is washed with 0.1% Tween 20/PBS, an HRP-luminescent substrate is added to each well and light intensity measured using a LJL Analyst plate luminometer.

IL-2

A 100 μL suspension of diluted human white blood cells contained within each well of a polystyrene 384-well plate (NalgeNunc) is stimulated to secrete IL-2 by treatment with a final concentration of 10 ng/mL phorbol 12-myristate 13-acetate (Sigma, P-1585) and 750 ng/mL ionomycin (Sigma, 1-0634). Various concentrations of each test compound are added at the time of stimulation. After 16-18 hours of incubation at 37° C. in a humidified incubator, the plate is centrifuged and the supernatant transferred to a white opaque polystyrene 384 well plate (NalgeNunc, Maxisorb) coated with an anti-IL-2 antibody (PharMingen, #555051). After a two-hour incubation, the plate is washed (Tecan PowerWasher 384) with PBS containing 0.1% Tween 20 and incubated for an additional one hour with another anti-IL-2 antibody that is biotin labeled (Endogen, M600B) and HRP coupled to strepavidin (PharMingen, #13047E). After the plate is washed with 0.1% Tween 20/PBS, an HRP-luminescent substrate is added to each well and light intensity measured using a LJL Analyst plate luminometer.

IL4 and IL-5

Analysis of IL-4 and IL-5 cytokine expression is performed using the BD PharMingen Cytometric 6 Bead Array system according to the manufacturer's instructions. Briefly, the supernatant from a buffy coat assay plate is incubated with the labeled cytokine detection bead cocktail. The samples are then washed, resuspended and read on the BD Pharmingen FACsCalibur flow cytometer. Data is then analyzed using the BD Pharmingen CBA 6 Bead Analysis software.

### EXAMPLE 1

### Parallel Signaling Pathways are Inhibited by Amoxapine and Paroxetine

Materials and Methods

Drugs

Stock solutions were made in DMSO for all drugs except amoxapine which was prepared in 0.1 mM MES (2-(N-morpholinoethanesulfonic acid) (Sigma) buffer. Stock solutions of phorbol myristate acetate (PMA) (100 μg/ml), and ionomycin (5 mg/ml) in DMSO were diluted in the culture media to produce final concentrations of PMA (10 ng/ml, 16.2 nM) and ionomycin (750 μg/ml, 1 μM).

Cells and Cell Lines

Fresh buffy coat preparations from donated human blood (Red Cross, Rhode Island) were used to isolate peripheral blood mononuclear cells (PBMCs) by Ficoll-Plaque (Pharmacia) layered centrifugation. T cells were purified from PBMCs using “Pan T cell Isolation Kit II—human”, (Miltenenyibiotec, Germany). A lymphoid leukemia T cell line (CCRF-CEM) was obtained from American Type Cell Culture (ATCC). All cells were grown in RPMI 1640 medium (Cellgro) supplemented with 10% serum (Gibco) and 1% Pen-Strep solution (Cellgro). For nuclear translocation studies, cells were grown in the serum-starved medium containing 0.1% serum.

ELISA for Cytokine Screening

Antibodies for enzyme linked immunosorbant assay (ELISA) were obtained from BD Pharmingen. Sandwich ELISA was done by standard procedure with some modifications. Buffy coat or isolated T cells were diluted with culture medium in 384 well plates containing compounds and inducer (PMA/ionomycin (PI)). The plates were incubated at 37° C. for 16-18 hours. After centrifugation the supernatant was removed and transferred to a 384-well plate containing capture antibody. The capture antibody was coated overnight (16-18 hours) at 4° C. and aspirated off before adding the supernatant. After incubation for two hours, plates were washed with PBS (0.1% Tween20), and detection antibodies added. Fluorescence intensity was measure with luciferase substrate (Amersham) by luminometer (LJL or Wallac).

Transactivation Assay

Reporter plasmids were transfected into CCRF-CEM cells using nucleofection (Nucleofector; AMAXA, Germany). Reporter plasmids expressing firefly luciferase (Luc) were purchased from Stratagene. pNFAT-Luc contains four NFAT binding sites; pGRE-Luc contains four GRE sites, and pAP1-Luc contains seven AP1 binding sites. The NFκB luciferase reporter, p(IL6κB)₃-50hu.IL6-luc+, contains three NFκB sites and was a generous gift of Dr. De Bosscher (University of Ghent, Belgium). 10⁷ cells suspended in 100 μL Amaxa ‘R’ Cell-line solution were transferred to a cuvette. One μL (1 μg/μL) of solution containing reporter plasmid (firefly luciferase) and control plasmid (pRL-TK-Renilla) (Renilla luciferase) (Promega) at a ratio of (10:1) were added to the cell suspension. Transfection was done with Amaxa Nucleofector using program T-14, which gave maximum efficiency with CCRF-CEM cells. After transfection the cells were suspended in 200 μL medium, allowed to recover for an hour, equal volume of medium containing 2× drugs were added and incubated at 37° C. for 30 min. Then the cells were stimulated with PI for another five hours. The luciferase activity of each of plasmid and control Renilla-plasmid were measured as per the procedure in Promega Luciferase assay kit.

Westerns Blot Assays

Purified primary human T-cells (10⁷ T-cells at 1×10⁶ cells/ml) were pretreated with various drugs for 30 min at 37° C. and then stimulated for 30 minutes with PMA and ionomycin. Cells were then pelleted and extracted with 2× protein load dye (Invitrogen, NP0008). Total cell lysates were boiled and centrifuged before loading. 10-15 μl of lysate (approximately 250,000 cells) per lane was run on a 10-12% Tris-Bis gel, or a 3-8% Tris-Acetate gel (precast from Invitrogen). Proteins were immunoblotted onto immobilon PVDF membrane (Millipore) for 30 min using an Owl Semi-Dry electroblotting system. Membranes were blocked with 4% milk for two hours and then incubated with appropriate primary antibodies, washed three times and then probed with secondary antibodies. Chemi-glow™ (Alpha Innotech) chemilluminescent detection solutions were added and image visualized and captured using an Alpha Imager 8900 (Alpha Innotech). NFAT1 was visualized using an antibody obtained from BD Transduction Laboratory (#610703). IκB-alpha was visualized using an antibody from Santa Cruz Biotechnology (#sc371). Mitogen activated protein kinases (MAPK) were visualized using the following antibodies obtained from Cell Signaling: ERK p44/p42 (phospho, #9101; total, #9102); p38 (phospho, #9211; total, #9212); and JNK/SAPK (phospho, #9251; total, #9252).

Translocation Assays

CCRF-CEM cells were grown in complete media (10% serum, RPMI 1640) to a density of 2×10⁵ cells/ml and then serum starved media (0.1% FBS, RPMI 1640) overnight for 16 hours. The cells were dropped onto poly L-lysine-coated glass coverslips (Fisher) and allowed to attach to the cover slip for 15 minutes. The cell-coated coverslips were preincubated with drug for 20 minutes and then stimulated for an hour with either 1×PMA+ionomycin or prednisolone in serum starved media. After incubation with drug plus stimulant, media was aspirated off and the cells were fixed for 15 minutes with 3.7% formaldehyde in PBS. The cells were washed three times with 1×PBS, 0.2% Triton, blocked twice for 15 minutes in “Superblock”™ (Pierce) and incubated for 30 minutes with primary antibody (1:5000 dilution). NFAT1 was visualized using an antibody obtained from BD Transduction Laboratory (#610703). NFκB (p65 component) was visualized using an antibody obtained from Santa Cruz Biotechnology (#sc-372). Coverslips were washed again three times before adding labeled secondary antibodies (Alexa Fluor™, Molecular Probes). Nuclei were labeled with DAPI (Sigma). Finally the coverslips were washed once with PBS/Triton and mounted with Fluoromount™ onto glass microscope slides for viewing under a Nikon fluorescent microscope. Translocation of transcription factors into nucleus was quantified by scoring of blinded slides.

Results

Amoxapine and Paroxetine Repress the NFAT Pathway

A consequence of T cell activation is an increase in intracellular calcium that activates calcineurin, a serine/threonine phosphatase. Calcineurin in turn dephosphorylates cytoplasmic NFAT triggering nuclear translocation of NFAT. In the nucleus, NFAT binds regulatory sites in the promoters of proinflammatory genes including TNFα contributing to their transcriptional induction. In our study we examined the effects of amoxapine and paroxetine on three stages of NFAT activation: i) dephosphorylation of NFAT protein, ii) translocation of NFAT to the nucleus and iii) activation of NFAT-dependent transcription.

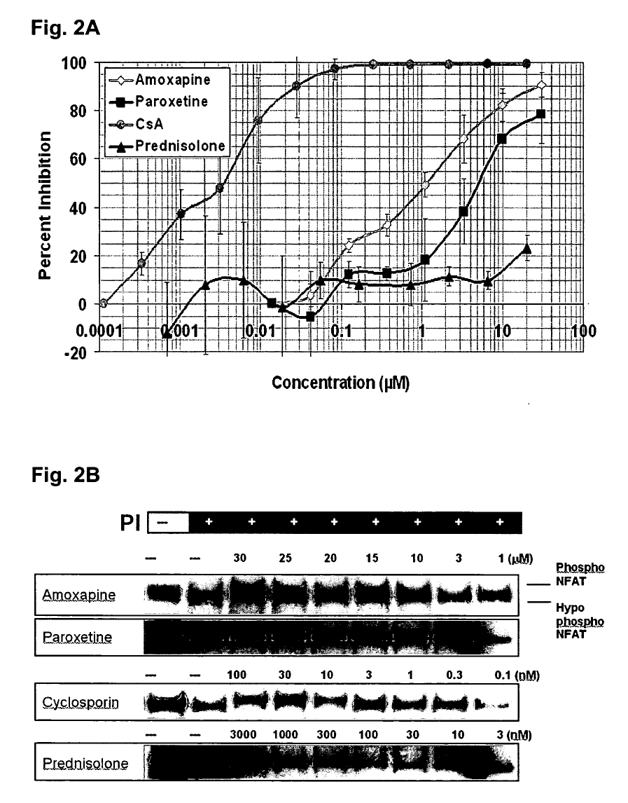

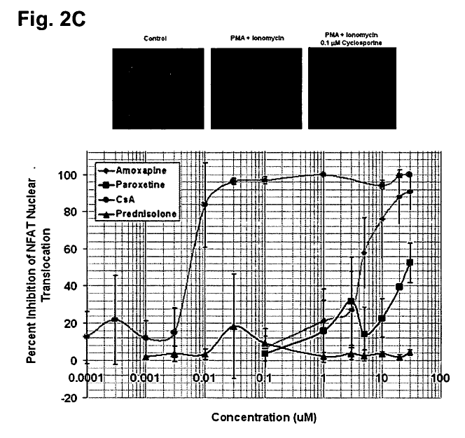

T cells were isolated from the buffy coat of male donors between the ages of 35 to 50 years. These T cells were activated in vitro with PMA/ionomycin (PI) for 30 minutes and the phosphorylation of NFAT analyzed by mobility shift on a western blot. The dephosphoryated NFAT in activated T cells moves with greater mobility in SDS PAGE and so produces a band shift. The results are shown in FIG. 2B. As expected, preincubation of cyclosporine, a direct inhibitor of calcineurin prevented a band shift below 3 nM. Similarly, preincubation with amoxapine (3 μM) and paroxetine (slight effect around 30 μM) prevented the band shift. Prednisolone had no effect on NFAT dephosphorylation up to 3 μM. The band shift transition concentrations observed for both cyclosporine and amoxapine are separated by 1000-fold, which closely matches their potency difference observed in the PI stimulated TNFα release assay.

The translocation of NFAT into the nucleus in PI-activated CCRF-CEM cells was tracked by immunofluorescence (FIG. 2C). Again as expected, cyclosporine inhibited the translocation of NFAT, generating an IC50 value of 5 nM, which closely parallels the behavior of cyclosporine in both the cytokine inhibition and NFAT band shift assays. Amoxapine and paroxetine also inhibited NFAT translocation, with IC50s of 4 μM and 30 μM respectively. Prednisolone was not active in this assay. The overall results of the NFAT translocation study agree with the rank order of compound potency observed in the cytokine and western blot analyses.

Finally, NFAT dependent transcription was measured by transient transfection of a NFAT reporter plasmid into CCRF-CEM cells and subsequent activation with PI. The results are shown in FIG. 2A. Cyclosporine was again effective at inhibiting PI-stimulated NFAT transcription with an IC50 of 5 nM, in agreement with the effect observed in the cytokines, band shift, and translocation assays. Amoxapine and paroxetine generated an IC50 of 2 μM and 9 μM respectively. Prednisolone showed no strong inhibition even at high doses.

Amoxapine and Paroxetine Repress the NF-κB Pathway

Like NFAT, NFκB is a critical regulatory transcription factor for the activation of proinflammatory cytokine genes. NFκB is sequestered in the cytoplasm in complex with IκB. Up on T cell activation, IκB is phosphorylated and degrades, freeing NFκB to translocate to the nucleus and activate genes involved in inflammation. We assessed the effect of amoxapine and paroxetine on the degradation of IκB, the translocation of NFκB to the nucleus, and NFκB-dependent transcriptional activation.

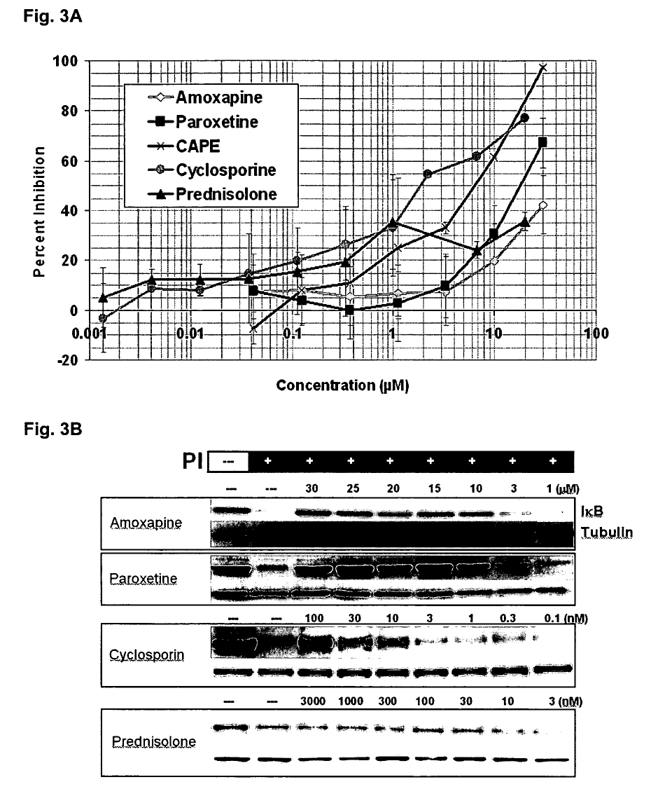

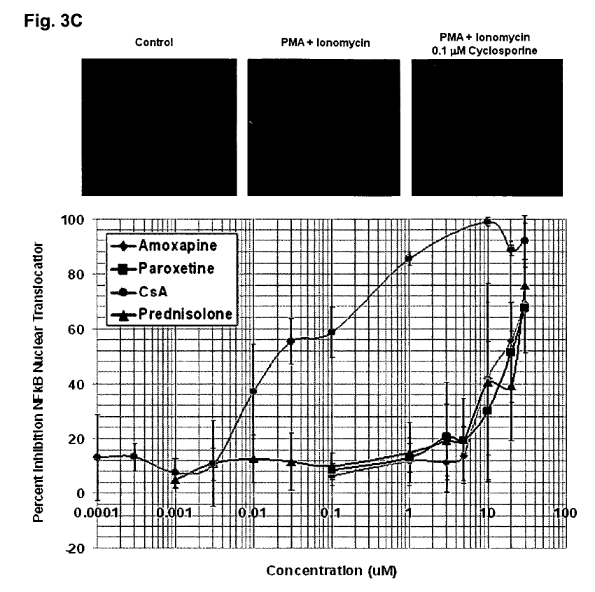

Primary T cells were activated in vitro with PI (30 min) and extracted for western blot analysis. Cyclosporine, amoxapine, and paroxetine stabilize IκB (FIG. 3B) but with different potencies. Cyclosporine was most potent, with effects starting at 30 nM. The effects of amoxapine and paroxetine began at 25 μM and 15 μM, respectively. This observation reverses the potency rank order observed for these compounds in TNFα inhibition assays. Predinosolone had no effect on IκB degradation.

Nuclear translocation of NFκB was assayed in activated CCRF-CEM cells by immunofluorescence using antibodies to the p65 component of NFκB. The results are shown in FIG. 3C. Again cyclosporine potently inhibited NFκB translocation with an IC50 of 20 nM. Amoxapine and paroxetine had nearly identical inhibitory curves, each generated an IC50 of 20 μM. Prednisolone had no effect on NFκB translocation.

NFκB-dependent transcription was measured by transient transfection of an NFκB reporter plasmid into CCRF-CEM cells and subsequent activation with PI. The results of this experiment are depicted in FIG. 3A. The NFκB inhibitor CAPE inhibited as expected but cyclosporine had little effect in the NFkB transcription assay up to 1 μM. Cyclosporine and amoxapine behave with similar effect at high concentration (IC50=20 μM), while paroxetine achieved a 40% inhibition at the highest concentration of 30 μM. Prednisolone showed 30% inhibition at 1 μM, which is consistent with reported glucocorticoid transrepression of NFκB transcription.

Amoxapine and Paroxetine Repress the MAP Kinase Pathway

T cell activation triggers multiple signal transduction pathways. In addition to NFAT and NFkB activation, the MAP kinase cascade is also activated. This cascade consists of three main arms that culminate in the activation of ERK, p38, and JNK. Some substrates of these MAP kinases include transcription factors such as ELK1, ERG, and AP1, which in turn regulate proinflammatory gene expression. We set out to track the activation of ERK, p38, and JNK in the presence of amoxapine or paroxetine.

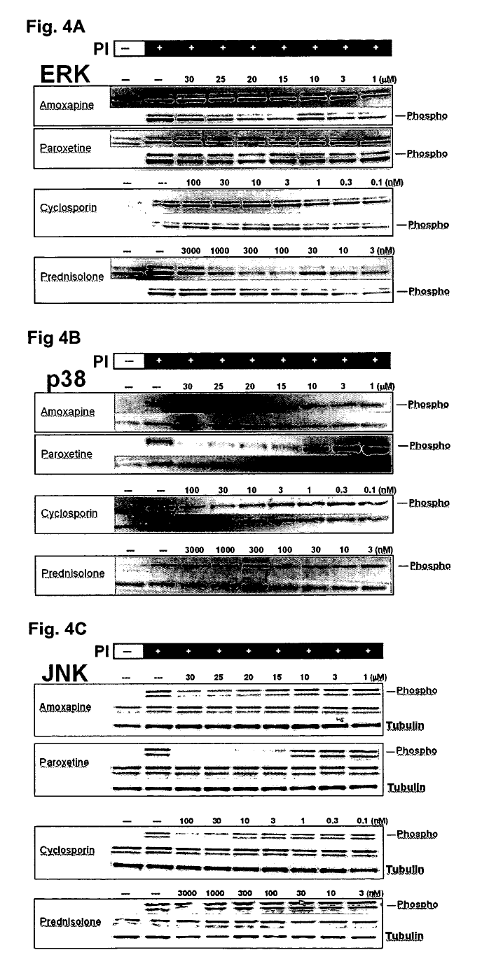

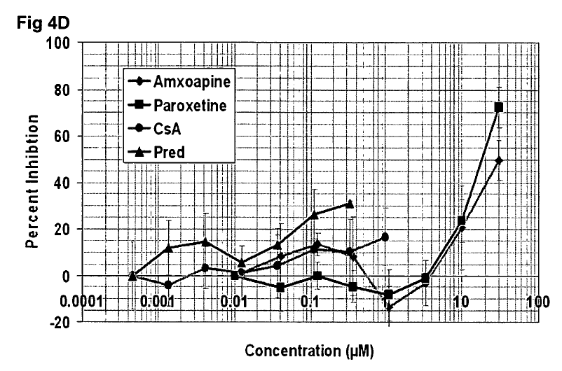

Purified primary T cells were activated for 30 minutes and extracted for western blot analysis. Activation of each MAPK was tracked using phosphospecific antibodies to a regulatory site on each type of MAPK, normalized by the measurement of total amounts of each MAPK species (FIGS. 4A-4C). Even the highest dose of cyclosporine (1 μM), prednisolone (3 μM), amoxapine (30 μM), and paroxetine (30 μM) tested were not effective in preventing phosphorylation of ERK1/2. In contrast, above 30 nM cyclosporine, there was evidence of some phospho-p38 inhibition. Paroxetine showed some inhibition of phospho-p38 above 10 μM, but amoxapine and prednisolone had no effect in the p38 assay. When JNK activation was analyzed, cyclosporine was observed to inhibit JNK activation above 30 nM. Both amoxapine and paroxetine showed similar inhibition above 10-20 μM. Prednisolone had no effect on JNK.

AP1-dependent transcription was measured by transient transfection of an AP1 reporter plasmid into CCRF-CEM cells and subsequent activation with PI (FIG. 4D). Cyclosporine showed little effect up to 1 μM, a level that is more than 100 times the concentration that produces near complete inhibition of TNF-PI. In contrast, amoxapine and paroxetine show similar inhibition curves with IC50 in the range of 20-30 μM, a level at which these drug have effect in the cytokine assay. Prednisolone at 300 nM generated 30% inhibition consistent with glucocorticoid transrepression observed for AP1 transcription.

Administration

In particular embodiments of any of the methods of the invention, the compounds are administered within 10 days of each other, within five days of each other, within twenty-four hours of each other, or simultaneously. The compounds may be formulated together as a single composition, or may be formulated and administered separately. One or both compounds may be administered in a low dosage or in a high dosage, each of which is defined herein. It may be desirable to administer to the patient other compounds, such as a corticosteroid, NSAID (e.g., naproxen sodium, diclofenac sodium, diclofenac potassium, aspirin, sulindac, diflunisal, piroxicam, indomethacin, ibuprofen, nabumetone, choline magnesium trisalicylate, sodium salicylate, salicylsalicylic acid, fenoprofen, flurbiprofen, ketoprofen, meclofenamate sodium, meloxicam, oxaprozin, sulindac, and tolmetin), COX-2 inhibitor (e.g., rofecoxib, celecoxib, valdecoxib, and lumiracoxib), or DMARD. Combination therapies of the invention are especially useful for the treatment of immunoinflammatory disorders in combination with other anti-cytokine agents or agents that modulate the immune response to positively effect disease, such as agents that influence cell adhesion, or biologics (i.e., agents that block the action of IL-6, IL-1, IL-2, IL-12, IL-15 or TNFα (e.g., etanercept, adelimumab, infliximab, or CDP-870). In this example (that of agents blocking the effect of TNFα), the combination therapy reduces the production of cytokines, etanercept or infliximab act on the remaining fraction of inflammatory cytokines, providing enhanced treatment.

Therapy according to the invention may be performed alone or in conjunction with another therapy and may be provided at home, the doctor's office, a clinic, a hospital's outpatient department, or a hospital. Treatment optionally begins at a hospital so that the doctor can observe the therapy's effects closely and make any adjustments that are needed, or it may begin on an outpatient basis. The duration of the therapy depends on the type of disease or disorder being treated, the age and condition of the patient, the stage and type of the patient's disease, and how the patient responds to the treatment. Additionally, a person having a greater risk of developing an inflammatory disease (e.g., a person who is undergoing age-related hormonal changes) may receive treatment to inhibit or delay the onset of symptoms.

Routes of administration for the various embodiments include, but are not limited to, topical, transdermal, and systemic administration (such as, intravenous, intramuscular, subcutaneous, inhalation, rectal, buccal, vaginal, intraperitoneal, intraarticular, ophthalmic or oral administration). As used herein, “systemic administration” refers to all nondermal routes of administration, and specifically excludes topical and transdermal routes of administration.

In combination therapy, the dosage and frequency of administration of each component of the combination can be controlled independently. For example, one compound may be administered three times per day, while the second compound may be administered once per day. Combination therapy may be given in on-and-off cycles that include rest periods so that the patient's body has a chance to recover from any as yet unforeseen side effects. The compounds may also be formulated together such that one administration delivers both compounds.

Formulation of Pharmaceutical Compositions

The administration of a combination of the invention may be by any suitable means that results in suppression of proinflammatory cytokine levels at the target region. The compound may be contained in any appropriate amount in any suitable carrier substance, and is generally present in an amount of 1-95% by weight of the total weight of the composition. The composition may be provided in a dosage form that is suitable for the oral, parenteral (e.g., intravenously, intramuscularly), rectal, cutaneous, nasal, vaginal, inhalant, skin (patch), or ocular administration route. Thus, the composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, or aerosols. The pharmaceutical compositions may be formulated according to conventional pharmaceutical practice (see, e.g., Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York).

Each compound of the combination may be formulated in a variety of ways that are known in the art. For example, the first agent (an agent that increases the signaling activity of a glucocorticoid receptor) and the second agent (i.e., the non-steroidal agent that reduces signaling activity of one or more of the NFκB, NFAT, AP-1 or Elk-1 pathway) may be formulated together or separately. Desirably, the first and second agents are formulated together for the simultaneous or near simultaneous administration of the agents. Such co-formulated compositions can include the two agents formulated together in the same pill, capsule, liquid, etc. It is to be understood that, when referring to the formulation of such combinations, the formulation technology employed is also useful for the formulation of the individual agents of the combination, as well as other combinations of the invention. By using different formulation strategies for different agents, the pharmacokinetic profiles for each agent can be suitably matched.

The individually or separately formulated agents can be packaged together as a kit. Non-limiting examples include kits that contain, e.g., two pills, a pill and a powder, a suppository and a liquid in a vial, two topical creams, etc. The kit can include optional components that aid in the administration of the unit dose to patients, such as vials for reconstituting powder forms, syringes for injection, customized IV delivery systems, inhalers, etc. Additionally, the unit dose kit can contain instructions for preparation and administration of the compositions. The kit may be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

Dosages

Generally, when administered to a human, the dosage of the non-steroidal agent that reduces signaling activity of one or more of the NFκB, NFAT, AP-1 or Elk-1 pathway will depend on the nature of the agent, and can readily be determined by one skilled in the art. Typically, such dosage is normally about 0.001 mg to 2000 mg per day, desirably about 1 mg to 1000 mg per day, and more desirably about 5 mg to 500 mg per day. Dosages up to 200 mg per day may be necessary.

When systemically administered to a human, the dosage of the agent that increases the signaling activity of a glucocorticoid receptor for use in the combination of the invention is normally about 0.1 mg to 1500 mg per day, desirably about 0.5 mg to 10 mg per day, and more desirably about 0.5 mg to 5 mg per day.

Administration of each drug in the combination can, independently, be one to four times daily for one day to one year, and may even be for the life of the patient. Chronic, long-term administration will be indicated in many cases.

Additional Applications

The compounds of the invention can be employed in immunomodulatory or mechanistic assays to determine whether other combinations, or single agents, are as effective as the combination in inhibiting secretion or production of proinflammatory cytokines or modulating immune response using assays generally known in the art, examples of which are described herein. For example, candidate compounds may be combined with an agent that increases the signaling activity of a glucocorticoid receptor or a non-steroidal agent that reduces the signaling activity of one or more of the NFκB, NFAT, AP-1 or Elk-1 pathway and applied to stimulated PBMCs. After a suitable time, the cells are examined for cytokine secretion or production or other suitable immune response. The relative effects of the combinations versus each other, and versus the single agents are compared, and effective compounds and combinations are identified.

The combinations of the invention are also useful tools in elucidating mechanistic information about the biological pathways involved in inflammation. Such information can lead to the development of new combinations or single agents for inhibiting inflammation caused by proinflammatory cytokines. Methods known in the art to determine biological pathways can be used to determine the pathway, or network of pathways affected by contacting cells stimulated to produce proinflammatory cytokines with the compounds of the invention. Such methods can include, analyzing cellular constituents that are expressed or repressed after contact with the compounds of the invention as compared to untreated, positive or negative control compounds, and/or new single agents and combinations, or analyzing some other metabolic activity of the cell such as enzyme activity, nutrient uptake, and proliferation. Cellular components analyzed can include gene transcripts, and protein expression. Suitable methods can include standard biochemistry techniques, radiolabeling the compounds of the invention (e.g., ¹⁴C or ³H labeling), and observing the compounds binding to proteins, e.g. using 2d gels, gene expression profiling. Once identified, such compounds can be used in in vivo models to further validate the tool or develop new anti-inflammatory agents.

### Other Embodiments

All publications, patent applications, and patents mentioned in this specification are herein incorporated by reference.

Various modifications and variations of the described method and system of the invention will be apparent to those skilled in the art without departing from the scope and spirit of the invention. Although the invention has been described in connection with specific desired embodiments, it should be understood that the invention as claimed should not be unduly limited to such specific embodiments. Indeed, various modifications of the described modes for carrying out the invention that are obvious to those skilled in the fields of medicine, immunology, pharmacology, endocrinology, or related fields are intended to be within the scope of the invention.

1. A composition comprising:
(a) an agent that increases glucocorticoid receptor signaling activity; and
(b) a non-steroidal agent that modulates the signaling activity of one or more signaling pathways selected from the NF-κB pathway, NFAT pathway, AP-1 pathway, and Elk-1 pathway such that proinflammatory cytokine secretion or production, or any other inflammatory response, is reduced,
wherein said agent that increases the signaling activity of a glucocorticoid receptor and said non-steroidal agent are present in amounts that, when administered to a mammal, are sufficient to reduce proinflammatory cytokine secretion or production.

2. The composition of claim 1, wherein said non-steroidal agent modulates the signaling activity of two or more signaling pathways.

3. The composition of claim 2, wherein said non-steroidal agent modulates the activity of three or more signaling pathways.

4. The composition of claim 1, wherein said proinflammatory cytokine is TNF-α.

5. The composition of claim 1, wherein said an agent that increases glucocorticoid receptor signaling activity is present in said composition in low dosage.

6. The composition of claim 1, wherein said non-steroidal agent is an agent that increases or decreases the expression level or biological activity of a signaling molecule such that the signaling activity of one or more signaling pathways selected from the NF-κB pathway, NFAT pathway, AP-1 pathway, and Elk-1 pathway is modulated.

7. The composition of claim 6, wherein said non-steroidal agent is an NF-κB pathway inhibitor, NFAT pathway inhibitor, AP-1 pathway inhibitor, or Elk-1 pathway inhibitor.

8. The composition of claim 1, wherein said non-steroidal agent is an antisense compound or RNAi compound that reduces the expression levels of a signaling molecule such that the signaling activity of one or more signaling pathways selected from the NF-κB pathway, NFAT pathway, AP-1 pathway, and Elk-1 pathway is modulated.

9. The composition of claim 1, wherein said non-steroidal agent is a dominant negative of a signaling molecule or an expression vector encoding said dominant negative such that the signaling activity of one or more signaling pathways selected from the NF-κB pathway, NFAT pathway, AP-1 pathway, and Elk-1 pathway is modulated.

10. The composition of claim 1, wherein said non-steroidal agent is an antibody that binds a signaling molecule and reduces the biological activity of said signaling molecule such that the signaling activity of one or more signaling pathways selected from the NF-κB pathway, NFAT pathway, AP-1 pathway, and Elk-1 pathway is modulated.

11. The composition of claim 10, wherein said biological activity is enzymatic activity, phosphorylation state, or binding activity.

12. The composition of claim 1, further comprising an additional therapeutic compound.

13. The composition of claim 12, wherein said additional therapeutic compound is selected from the group consisting of an NSAID, small molecule immunomodulator, COX-2 inhibitor, DMARD, biologic, xanthine, anticholinergic compound, beta receptor agonist, bronchodilator, non-steroidal calcineurin inhibitor, vitamin D analog, psoralen, retinoid, and 5-amino salicylic acid.

14. The composition of claim 13, wherein said NSAID is ibuprofen, diclofenac, or naproxen.

15. The composition of claim 13, wherein said COX-2 inhibitor is rofecoxib, celecoxib, valdecoxib, or lumiracoxib.

16. The composition of claim 13, wherein said biologic is adelimumab, etanercept, or infliximab.

17. The composition of claim 13, wherein said DMARD is methotrexate or leflunomide.

18. The composition of claim 13, wherein said xanthine is theophylline.

19. The composition of claim 13, wherein said anticholinergic compound is ipratropium or tiotropium.

20. The composition of claim 13, wherein said beta receptor agonist is ibuterol sulfate, bitolterol mesylate, epinephrine, formoterol fumarate, isoproteronol, levalbuterol hydrochloride, metaproterenol sulfate, pirbuterol scetate, salmeterol xinafoate, or terbutaline.

21. The composition of claim 13, wherein said non-steroidal calcineurin inhibitor is cyclosporine, tacrolimus, pimecrolimus, or ISAtx247.

22. The composition of claim 13, wherein said vitamin D analog is calcipotriene or calcipotriol.

23. The composition of claim 13, wherein said psoralen is methoxsalen.

24. The composition of claim 13, wherein said retinoid is acitretin or tazoretene.

25. The composition of claim 13, wherein said 5-amino salicylic acid is mesalamine, sulfasalazine, balsalazide disodium, or olsalazine sodium.

26. The composition of claim 1, wherein said composition is formulated for topical administration.

27. The composition of claim 1, wherein said composition is formulated for systemic administration.

28. A method for treating, preventing, or reducing an immunoinflammatory disorder, said method comprising administering to a mammal a combination of:
(a) an agent that increases the signaling activity of a glucocorticoid receptor; and
(b) a non-steroidal agent that modulates the signaling activity of one or more signaling pathways selected from the NF-κB pathway, NFAT pathway, AP-1 pathway, and Elk-1 pathway such that proinflammatory cytokine secretion or production, or any other inflammatory response, is reduced,
wherein the first and second agents are administered simultaneously or within 28 days of each other, in amounts that together are sufficient to treat, prevent, or reduce said immunoinflammatory disorder.

29. The method of claim 28, wherein said non-steroidal agent modulates the signaling activity of two or more signaling pathways.

30. The method of claim 28, wherein said non-steroidal agent modulates the signaling activity of three or more signaling pathways.

31. The method of claim 28, wherein said combination reduces proinflammatory cytokine release or production.

32. The method of claim 31, wherein said proinflammatory cytokine is TNF-α.

33. The method of claim 28, wherein said agent that increases the signaling activity of the glucocorticoid receptor is present in said composition in low dosage.

34. The method of claim 28, wherein said non-steroidal agent is an agent that increases or decreases the expression level or biological activity of a signaling molecule such that the signaling activity of one or more signaling pathways selected from the NF-κB pathway, NFAT pathway, AP-1 pathway, and Elk-1 pathway is modulated.

35. The method of claim 28, wherein said non-steroidal agent is an NF-κB pathway inhibitor, NFAT pathway inhibitor, AP-1 pathway inhibitor, or Elk-1 pathway inhibitor.

36. The method of claim 28, wherein said non-steroidal agent is an antisense compound or RNAi compound that reduces the expression levels of a signaling molecule such that the signaling activity of one or more signaling pathways selected from the NF-κB pathway, NFAT pathway, AP-1 pathway, and Elk-1 pathway is modulated.

37. The method of claim 28, wherein said non-steroidal agent is a dominant negative of a signaling molecule or an expression vector encoding said dominant negative such that the signaling activity of one or more of the signaling pathways selected from the NF-κB pathway, NFAT pathway, AP-1 pathway, and Elk-1 pathway is modulated.

38. The method of claim 28, wherein said non-steroidal agent is an antibody that binds a signaling molecule and reduces the biological activity of said signaling molecule such that the signaling activity of one or more of the signaling pathways selected from the group consisting of the NF-κB pathway, said NFAT pathway, said AP-1 pathway, or said Elk-1 pathway is modulated.

39. The method of claim 38, wherein said biological activity is enzymatic activity, phosphorylation state, or binding activity.

40. The method of claim 28, further comprising administering to said mammal an additional therapeutic compound.

41. The method of claim 40, wherein said additional therapeutic compound is selected from the group consisting of an NSAID, small molecule immunomodulator, COX-2 inhibitor, DMARD, biologic, xanthine, anticholinergic compound, beta receptor agonist, bronchodilator, non-steroidal calcineurin inhibitor, vitamin D analog, psoralen, retinoid, and 5-amino salicylic acid.

42. The method of claim 41, wherein said NSAID is ibuprofen, diclofenac, or naproxen.

43. The method of claim 41, wherein said COX-2 inhibitor is rofecoxib, celecoxib, valdecoxib, or lumiracoxib.

44. The method of claim 41, wherein said biologic is adelimumab, etanercept, or infliximab.

45. The method of claim 41, wherein said DMARD is methotrexate or leflunomide.

46. The method of claim 41, wherein said xanthine is theophylline.

47. The method of claim 41, wherein said anticholinergic compound is ipratropium or tiotropium.

48. The method of claim 41, wherein said beta receptor agonist is ibuterol sulfate, bitolterol mesylate, epinephrine, formoterol fumarate, isoproteronol, levalbuterol hydrochloride, metaproterenol sulfate, pirbuterol scetate, salmeterol xinafoate, or terbutaline.

49. The method of claim 41, wherein said non-steroidal calcineurin inhibitor is cyclosporine, tacrolimus, pimecrolimus, or ISAtx247.

50. The method of claim 41, wherein said vitamin D analog is calcipotriene or calcipotriol.

51. The method of claim 41, wherein said psoralen is methoxsalen.

52. The method of claim 41, wherein said retinoid is acitretin or tazoretene.

53. The composition of claim 41, wherein said 5-amino salicylic acid is mesalamine, sulfasalazine, balsalazide disodium, or olsalazine sodium.

54. The method of claim 28, wherein said agent that increases the signaling activity of the glucocorticoid receptor and said non-steroidal agent are administered within 14 days of each other.

55. The method of claim 54, wherein said agent that increases the signaling activity of the glucocorticoid receptor and said non-steroidal agent are administered within 7 days of each other.

56. The method claim 55, wherein said agent that increases the signaling activity of the glucocorticoid receptor and said non-steroidal agent are administered within 1 day of each other.

57. The method of of claim 28, wherein said agent that increases the signaling activity of the glucocorticoid receptor, said non-steroidal agent, or both are administered topically or systemically.

58. A method of reducing the release from or production of inflammatory cytokines in inflammatory cells, comprising contacting inflammatory cells with an agent that increases the signaling activity of the glucocorticoid receptor and a non-steroidal agent that modulates the signaling activity of one or more signaling pathways selected from the NF-κB pathway, NFAT pathway, AP-1 pathway, and Elk-1 pathway such that proinflammatory cytokine secretion or production, or any other inflammatory response, is reduced.

59. A method for identifying a combination that may be useful for the treatment, prevention, or reduction of an immunoinflammatory disorder, said method comprising the steps of:
(a) contacting inflammatory cells in vitro with an agent that increases the signaling activity of the glucocorticoid receptor and a candidate compound; and
(b) determining whether the combination of said agent that increases the signaling activity of the glucocorticoid receptor and said candidate compound reduces proinflammatory cytokine release from or production in said cells relative to proinflammatory cytokine release from or production in cells contacted with said agent that increases the signaling activity of the glucocorticoid receptor but not contacted with the candidate compound, wherein a reduction in proinflammatory cytokine release or production identifies the combination as a combination useful for the treatment, prevention, or reduction of an immunoinflammatory disorder.

60. The method of claim 59, wherein said cells are T cells.

61. A method for identifying a candidate compound useful for the treatment, prevention, or reduction of an immunoinflammatory disorder, said method comprising the steps of:
(a) providing inflammatory cells having reduced glucocorticoid receptor signaling activity;
(b) contacting said cells with a candidate compound; and
(c) determining whether said candidate compound reduces cytokine release from or production in said cells relative to cells not contacted with said candidate compound, wherein a reduction in cytokine release or production identifies the candidate compound as a compound useful for the treatment, prevention, or reduction of an immunoinflammatory disorder.

62. A method for identifying a combination that may be useful for the treatment of an immunoinflammatory disorder, said method comprising the steps of:
(a) contacting inflammatory cells in vitro with an agent that increases the signaling activity of the glucocorticoid receptor and a candidate compound; and
(b) determining whether the combination of said agent that increases the signaling acitivity of the glucocorticoid receptor and said candidate compound reduces cytokine release from or production in said inflammatory cells relative to cytokine release or production from cells contacted with said agent that increases the signaling activity of the glucocorticoid receptor but not contacted with said candidate compound, wherein a reduction in cytokine release or production identifies the combination as a combination useful for the treatment, prevention, or reduction of an immunoinflammatory disorder.

63. A method for identifying a compound useful for the treatment, prevention, or reduction of an immunomodulatory disorder, said method comprising the steps of:
(a) providing inflammatory cells engineered to have modulated signaling activity in one or more signaling pathways selected from the NF-κB pathway, NFAT pathway, AP-1 pathway, and Elk-1 pathway;
(b) contacting said cells with a candidate compound; and
(c) determining whether said candidate compound reduces proinflammatory cytokine release from or production in said cells relative to cells not contacted with said candidate compound, wherein a reduction in cytokine release or production identifies said candidate compound as a compound useful for the treatment, prevention, or reduction of an immunoinflammatory disorder.

64. A method for identifying a combination useful for the treatment, prevention, or reduction of an immunoinflammatory disorder, said method comprising the steps of:
(a) identifying a compound that modulates the signaling activity of one or more signaling pathways selected from the NF-κB pathway, NFAT pathway, AP-1 pathway, and Elk-1 pathway such that proinflammatory cytokine secretion or production, or any other inflammatory response, is reduced;
(b) contacting inflammatory cells in vitro with an agent that increases the signaling activity of the glucocorticoid receptor and the compound identified in step (a); and
(c) determining whether the combination of said agent that increases the signaling activity of the glucocorticoid receptor and the compound identified in step (a) reduces proinflammatory cytokine release from or production in said cells relative to cells contacted with said agent that increases the signaling activity of the glucocorticoid receptor but not contacted with the compound identified in step (a) or contacted with the compound identified in step (a) but not contacted with said agent that increases the signaling activity of the glucocorticoid receptor, wherein a reduction in proinflammatory cytokine release or production identifies the combination as a combination useful for the treatment, prevention, or reduction of an immunoinflammatory disorder.

65. A method for identifying a combination useful for the treatment, prevention, or reduction of an immunoinflammatory disorder, said method comprising the steps of:
(a) identifying a compound that modulates the signaling activity of one or more signaling pathways selected from the NF-κB pathway, NFAT pathway, AP-1 pathway, and Elk-1 pathway such that proinflammatory cytokine secretion or production, or any other inflammatory response, is reduced;
(b) contacting inflammatory cells in vitro with an agent that increases the signaling activity of a glucocorticoid receptor and the compound identified in step (a); and
(c) determining whether the combination of said agent that increases the signaling activity of the glucocorticoid receptor and said compound identified in step (a) reduces proinflammatory cytokine release from or production in said cells relative to cytokine release from or production in cells contacted with said agent that increases the signaling activity of the glucocorticoid receptor but not contacted with the compound identified in step (a) or contacted with the compound identified in step (a) but not contacted with said agent that increases the signaling activity of the glucocorticoid receptor, wherein a reduction in proinflammatory cytokine release identifies the combination as useful for the treatment, prevention, or reduction of an immuno-inflammatory condition.

66. A kit, comprising:
(i) a composition comprising
(a) an agent that increases the signaling activity of the glucocorticoid receptor; and
(b) a non-steroidal agent that modulates the signaling activity of one or more signaling pathways selected from the NF-κB pathway, NFAT pathway, AP-1 pathway, and Elk-1 pathway such that proinflammatory cytokine secretion or production, or any other inflammatory response, is reduced; and
(ii) instructions for administering said composition to a patient diagnosed with an immunoinflammatory disorder.

67. A kit, comprising:
(i) an agent that increases the signaling activity of the glucocorticoid receptor;
(ii) a non-steroidal agent that modulates the signaling activity of one or more signaling pathways selected from the NF-κB pathway, NFAT pathway, AP-1 pathway, and Elk-1 pathway such that proinflammatory cytokine secretion or production, or any other inflammatory response, is reduced; and
(iii) instructions for administering said agent that increases the signaling activity of the glucocorticoid receptor and said non-steroidal agent to a patient diagnosed with an immunoinflammatory disorder.

68. A kit comprising:
(i) an agent that increases the signaling activity of the glucocorticoid receptor; and
(ii) instructions for administering said agent that increases the signaling activity of the glucocorticoid receptor and a non-steroidal agent that modulates the signaling activity of one or more signaling pathways selected from the NF-κB pathway, NFAT pathway, AP-1 pathway, and Elk-1 pathway such that proinflammatory cytokine secretion or production, or any other inflammatory response, is reduced to a patient diagnosed with an immunoinflammatory disorder.

69. A kit comprising:
(i) a non-steroidal agent that modulates the signaling activity of one or more signaling pathways selected from the NF-κB pathway, NFAT pathway, AP-1 pathway, and Elk-1 pathway such that proinflammatory cytokine secretion or production, or any other inflammatory response, is reduced; and
(ii) instructions for administering said agent and an agent that increases the signaling activity of the glucocorticoid receptor to a patient diagnosed with an immunoinflammatory disorder.
